(19) United States
(12) Patent Application Publication
(10) Pub. No.: US 2018/0140737 A1
(43) Pub. Date: May 24, 2018

(54) HAEMOSTATIC MATERIAL

(71) Applicant: HAEMOSTATIX LIMITED, Nottingham, Nottinghamshire (GB)

(72) Inventors: Renata ZBOZIEN, Nottingham, Nottinghamshire (GB); John Benjamin NICHOLS, Nottingham, Nottinghamshire (GB)

(21) Appl. No.: 15/572,908

(22) PCT Filed: May 11, 2016
(86) PCT No.: PCT/GB2016/051357

§ 371 Date: Nov. 9, 2017

(30) Foreign Application Priority Data
May 11, 2015 (GB) 1508020.3

(51) Int. Cl. A61L 15/28; A61L 15/32; A61L 15/44; C07K 17/12

(52) U.S. Cl. CPC A61L 15/28; A61L 15/32; A61L 15/44; A61L 2400/04; A61L 2300/232; A61L 2300/252; A61L 2300/418; C07K 17/12

(57) ABSTRACT

Haemostatic materials are described, particularly haemostatic materials comprising an oxidised cellulose substrate covalently immobilised to a plurality of fibrinogen-binding peptides. Methods are described for covalently attaching fibrinogen binding peptides to oxidised cellulose substrates ad other substrates that have carboxyl groups on their surface.

This invention relates to haemostatic materials, such as wound dressings, and methods of forming such materials. The invention also relates to methods of conjugating peptides to substrates.

Oxidised cellulose fabrics are bioresorbable and absorbent fabrics that have long been used in medical applications. Their beneficial properties include high absorbability, antibacterial and antiviral properties, and non-toxic and anti-adhesive effects.

Due to its ability to initiate or accelerate blood coagulation at a site where it is applied, oxidised cellulose can be used as a haemostatic material. An example of commercially available oxidised cellulose fabric is Surgicel® Nu-Knit® (manufactured by Ethicon Inc).

However, oxidised cellulose fabrics have several disadvantages including poor haemostatic properties, low biodegradability and low pH which can inactivate acid-sensitive proteins such as thrombin, albumin and globulin.

Efforts to modify oxidised cellulose and improve its properties have largely focussed on neutralising the acidity of the material. European patent EP 0659440 B1, for instance, describes treating oxidised cellulose with calcium or a combination of calcium and sodium or potassium. However, there is a desire to further improve the haemostatic properties of oxidised cellulose.

SPOT synthesis is an established method of solid phase peptide synthesis on cellulose membranes, and is commonly used to prepare peptide arrays (Hilpert, K., Winkler D. F. H, Hancock, R. E. W. (2007) Cellulose-bound Peptide Arrays: Preparation and Applications, Biotechnology and Genetic Engineering Reviews, 24: 1, 31-106). Cellulose is a polysaccharide with free hydroxyl groups. To make the cellulose suitable for synthesis of peptide, it is necessary to change the functionalisation from hydroxyl to more reactive amino groups. The easiest and often utilised derivatisation of cellulose membranes is the esterification using Fmoc β-alanine or Fmoc-Gly, and N,N′- diisopropyl carbodiimide.

However, the structure of oxidised cellulose differs from cellulose in that an oxidised cellulose substrate has carboxyl groups on its surface.

Oxidised cellulose is not suitable for conventional SPOT synthesis of peptide [symbol] peptides with free N-termini. This is because reactions may occur between the amino group of amino acids and carboxyl groups of the oxidised cellulose.

A further disadvantage of SPOT synthesis is the increased risk of epimerisation at all coupling stages due to repeated solid support-bound carboxyl activation.

The applicant has devised a more effective method of conjugating peptides to substrates such as oxidised cellulose and has surprisingly found that if fibrinogen binding peptides are covalently attached to oxidised cellulose substrates the haemostatic properties are improved considerably.

In a broad sense, the invention concerns covalent immobilisation of peptides, such as fibrinogen binding peptides, to a substrate and methods of covalently immobilising such peptides to a substrate.

Methods of the invention involve providing a moiety comprising a peptide and a first reactive group; providing a substrate with a second reactive group; and reacting the first reactive group with the second reactive group to covalently link the peptide to the substrate.

This approach contrasts with conventional SPOT synthesis, in which a peptide is synthesised in a carboxyl to amino direction whilst the C-terrninus is immobilised to the substrate. In other words, SPOT peptide synthesis occurs on the substrate. Conventional SPOT synthesis may be suitable for making peptide arrays on cellulose substrates, but it may be impractical and uneconomical for attaching peptides to substrates, such as wound dressings. For example, synthesising the peptide first, before conjugating the peptide to the substrate, may make it easier to characterise and control the purity of the material.

Methods of the invention encompass modifications to SPOT synthesis which are applicable to substrates having carboxyl groups, such as oxidised cellulose, for which SPOT synthesis may not be suitable. Examples of other substrates that have carboxyl groups include starch, glycogen, dextran, hemi-cellulose, pectin, hyaluronic acid, chitosan, gelatin, collagen and silk.

According to the invention, there is provided a method of covalently immobilising a peptide to a substrate, comprising: providing a moiety, wherein each moiety comprises a peptide and a first reactive group (or a moiety reactive group) in the form of a carbo [symbol] group; providing a substrate comprising a second reactive group (or a substrate reactive group) in the form of a carboxyl group; and reacting the first reactive group with the second reactive group to covalently immobilise the peptide to the substrate.

In preferred embodiments, the substrate is, or comprises, a cellulose-based substrate such as oxidised cellulose, most preferably regenerated oxidised cellulose.

The moiety preferably comprises a fibrinogen-binding peptide.

Preferably, the method involves covalently immobilising a plurality of peptides to the substrate.

According to the invention, there is provided a method of making a haemostatic material, or agent, comprising covalently immobilising a plurality of fibrinogen binding peptides to an oxidised cellulose substrate.

In some embodiments, the method comprises: providing a moiety comprising a fibrinogen-binding peptide and a first reactive group in the form of a carboxyl-reactive group; providing an oxidised cellulose substrate comprising a second reactive group in the form of a carboxyl group; and reacting the first reactive group with the second reactive group to covalently immobilise the peptide to the substrate.

Preferably, the first reactive group is an amino group, such that reaction of the first reactive group with the second reactive group forms an amide bond.

In a preferred embodiment, the peptide is covalently immobilised to the substrate via the C-terminus of the peptide.

According to the invention, there is provided a method of covalently immobilising a peptide to a substrate, comprising: providing a moiety comprising a peptide and a first reactive group in the form of a carboxyl-reactive group linked via the C-terminus of the peptide; providing a substrate comprising a second reactive group in the form of a carboxyl group; and reacting the first reactive group with the second reactive group to covalently immobilise each peptide to the substrate, such that the peptide is covalently immobilised to the substrate via its C-terminus.

The moiety may comprise a non-peptide portion, or linker. The non-peptide [symbol] provide the first reactive group. The non-peptide portion of the moiety may be covalently linked to the α-carbonyl group at the C-terminus of the peptide. Without the non-peptide portion providing the first reactive group, the peptide may not otherwise be able to react with the second reactive group on the substrate. The non-peptide portion may thus function as an adaptor. When the first and second reactive groups react, the non-peptide portion may form at least part of a spacer between the substrate and the peptide.

The non-peptide portion may be covalently attached to the peptide through an amide bond.

The moiety may comprise the following structure:

The non-peptide portion of the moiety may comprise a straight chain group, suitably of formula —(CH₂)ₐ—, wherein a is 1-20, preferably 1-15, 1-10, 1-5, or 2-4.

In a particularly preferred embodiment, the moiety comprises the following structure:

Where a=1-20, preferably 1-15, 1-10, 1-5, or 2-4.

Preferably the moiety has been synthesised by solid phase peptide synthesis, for example using standard Fmoc chemistry or Boc chemistry. The method of the invention may comprise a step of synthesising the moiety. Advantageously, the moiety is synthesised prior to its attachment to the substrate, rather than being synthesised in situ, on the substrate.

Reactive groups on the peptide of the moiety may be protected by protecting groups such as T-boc or F-MOG. Arginine side chains may be protected by 2,2,4,6,7-pentamethyldihydrobenzofuran-5-sulfonyl (Pbf) group or Pmc. Advantageously, only the first reactive group of the moiety may react with the second reactive group of the substrate. For example, an amino group on the N-terminus of the peptide, or any carboxyl-reactive amino acid side chains may be prevented from reacting with the first reactive group of the substrate. This is particularly advantageous if it is desired for the peptide to be conjugated to the substrate via its C-terminus, and for the N-terminus of the peptide to t [symbol] ligands.

In some embodiments, the N-terminus may not necessarily be protected, particularly if itis desirable for the N-terminal amino group of the peptide to react with the carboxyl group of the substrate, resulting in the peptide being bound to the substrate via its N-terminus.

The peptide may be deprotected once it has been covalently immobilised to the substrate.

In other embodiments, the peptide may be bound to the substrate through a side chain, for example through the side chain of a lysine residue. So, the first reactive group may be a side chain of an amino acid in the peptide.

In some embodiments, the substrate is modified (or activated) by reacting carboxyl groups of the substrate with modifying groups. The modifying groups may introduce spacers such that the second reactive groups are presented, or positioned, at the end of the spacers. This may improve accessibility of the second reactive group to the moiety during conjugation.

Examples of suitable spacers include peptide spacers. As used herein the term “peptide spacer” encompasses a spacer of one or more amino acid residues. In some embodiments, the peptide spacer is 1-5, or 1-2 amino acid residues in length. Examples of suitable peptide spacers include glycine, glycine-glycine, β-alanine, L-lysine or ϵ-aminohexanoic acid.

The method may comprise modifying the substrate by reacting the carboxyl groups on the substrate with modifying groups.

Preferably, each modifying group has a first reactive group, which is a carboxyl-reactive group and a second reactive group which is a carboxyl group. Preferably, the first reactive group of the modifying group is an amino group. The amino group may thus react with the carboxyl-group of the carrier to form an amide bond. So, although the second reactive group may be the same functional group that was present on the substrate before its modification, the introduction of a spacer may allow that functional group to be presented in a more accessible configuration.

Before modification, the substrate may comprise the following structure:

The modified substrate may comprise the following structure:

Once the modifying group has reacted with the carboxyl group on the substrate, it may provide a carboxyl-group for reaction with the first reactive group of the moiety, which may lead to formation of the amide bond.

For example, if the modifying group comprises two glycine residues, the modified substrate may comprise the following structure:

If the modifying group comprises 6-aminohexanoic acid, the modified substrate may comprise the following structure:

According to the invention, there is provided a method of covalently immobilising a peptide to a substrate, comprising: providing a moiety comprising a peptide and a first reactive group; providing a modified substrate comprising a second reactive group formed by modifying a carboxyl group of the substrate; and reacting the first reactive group with the second reactive group to covalently immobilise the peptide to the substrate.

In some embodiments, the method comprises: providing a moiety comprising a fibrinogen-binding peptide and a first reactive group; providing a modified oxidised cellulose substrate comprising a second reactive group formed by modifying a carboxyl group of the substrate; and reacting the first reactive group with the second reactive group to covalently immobilise the peptide to the substrate.

In some embodiments, the first reactive group is a carboxyl group, optionall [symbol] group at the C-terminus of the peptide. In other embodiments, the first reactive group may be linked via the C-terminus of the peptide.

According to the invention, there is provided a method of covalently immobilising a peptide to a substrate, comprising: providing a moiety comprising a peptide and a first reactive group, in which the first reactive group is the carboxyl group at the C-terminus of the peptide, or in which the first reactive group is linked via the C-terminus of the peptide; providing a modified substrate comprising a second reactive group formed by modifying a carboxyl group of the substrate; and reacting the first reactive group with the second reactive group to covalently immobilise the peptide to the substrate, such that the peptide is covalently attached to the substrate via its C-terminus.

The second reactive group may be a non-carboxyl group, preferably a carboxyl-reactive group, most preferably an amino group. The second reactive group may be for reaction with a carboxyl group of the moiety, such as a carboxyl group at the C-terminus of the peptide on a side chain of an amino acid residue of the peptide.

The carboxyl group of the substrate may have been modified (or activated) by reacting it with a modifying group. The method may thus comprise modifying the substrate. The modification may lead to incorporation of a spacer, with the second reactive group of the substrate positioned at the end of the spacer. The spacer may be covalently attached to the via an amide bond.

The modified substrate may comprise the following structure:

The spacer may comprise a straight chain group, suitably of formula —(CH₂)ₐ—, wherein a is 1-20, preferably 1-15, 1-10, 1-5, or 2-4

The modifying group may comprise a first carboxyl reactive group and a second carboxyl reactive group, wherein the first and second carboxyl reactive groups are preferably amino groups.

The modifying group may comprise the following structure:

H₂N—(CH₂)ₐ—NH₂

where a is 1-20, preferably 1-15, 1-10 or 1-6.

The modifying group may thus comprise a diamine molecule, such as ethylenediamine, 1,6-hexanediamine or 1,4-butadiamine.

For example, if the modifying group comprises ethylenediamine, the modified substrate may comprise the following structure:

The invention provides a peptide covalently bound to a substrate, obtainable by a method of the invention.

The invention provides a method for modifying (or activating) a substrate, comprising reacting a carboxyl group on the substrate with a modifying group, in which the modifying group comprises a first reactive group, preferably a carboxyl-reactive group, most preferably an amino group; and a second reactive group, preferably an amino reactive group, most preferably a carboxyl group.

The modifying group may comprise a peptide.

Before modification, the substrate may comprise the following structure:

The modified substrate may be described as activated or derivatised.

The invention provides a modified substrate, preferably a modified oxidised cellulose substrate, comprising the following structure:

where X is an amino-reactive group, preferably a carboxyl group.

Preferably, the spacer is attached via an amide bond, So, the substrate may comprise the following structure:

where X is an amino-reactive group, preferably a carboxyl group. So, the modified substrate may comprise the following structure:

The spacer may comprise a peptide spacer, as described herein.

The invention provides a method for modifying (or activating) a substrate, comprising reacting a carboxyl group on the substrate with a modifying group, in which the modifying group comprises a first reactive group, preferably a carboxyl-reactive group, most preferably an amino group, and a second reactive group, preferably a carboxyl-reactive group, most preferably an amino group.

Each modifying group may comprise the following structure:

H₂N—(CH₂)ₐ—NH₂

where a is 1-20, preferably 1-15, 1-10 or 1-6.

The invention provides a modified substrate, preferably a modified oxidised cellulose substrate, comprising the following structure:

where X is a carboxyl-reactive group, preferably an amino group.

Preferably, the spacer is attached via an amide bond. So, the modified substrate may comprise the following structure:

where X is a carboxyl-reactive group, preferably an amino group. So, the modified substrate may comprise the following structure:

The spacer may comprise the group —(CH₂)ₐ—, where a is 1-20, preferably 1-15, 1-10 or 1-6.

The invention provides a modified substrate obtainable by a method of the invention.

The invention also concerns a material comprising a substrate covalently be [symbol] peptide. The substrate preferably comprises carboxyl groups, and the peptide may be bound to the substrate by reacting a moiety comprising the peptide, with a carboxyl group on the substrate.

Preferably, the substrate is, or comprises, oxidised cellulose. Preferably, the peptide is a fibrinogen-binding peptide. Preferably the substrate is covalently bound to a plurality of peptides.

According to the invention, there is provided a haemostatic material (or agent) comprising an oxidised cellulose substrate covalently immobilised to a plurality of fibrinogen-binding peptides.

Preferably, the peptide is immobilised to the substrate via a carbonyl group of the substrate. For example, each peptide may be immobilised to the substrate by reacting the peptide, or a moiety comprising the peptide, with a carboxyl group on the substrate. The peptide may be attached to the substrate via an amide bond.

Advantageously, using a carboxyl group on the substrate as point of attachment for the peptide may modify the acidic properties of the substrate and improve interactions with proteins such as thrombin and fibrinogen.

In preferred embodiments, the peptide is immobilised to the substrate via a spacer. A spacer may increase accessibility to ligands. In the case of fibrinogen binding peptides, it may increase accessibility to fibrinogen and improve haemostatic activity.

The material may comprise the following structure:

In preferred embodiments, the peptide is immobilised to the substrate via its C-terminus. The N-terminus of the peptide may thus be accessible and available for interaction with ligands. The peptide may be attached to the substrate via the main chain a-carbonyl group at the C-terminus.

So, the material may comprise the following structure:

Alternatively, the peptide may be bound to substrate via its N-terminus. The material may thus comprise the following structure:

Alternatively, the peptide could be bound via a side chain of one of its amino acid residues.

Preferably, the spacer is attached to the peptide by an amide bond. So, the material may comprise one or both of the following structures:

Preferably, the spacer is attached to the substrate by an amide bond

In preferred embodiments, the spacer comprises a peptide spacer. Examples of suitable peptide spacers include glycine, glycine-glycine, β-alanine, L-lysine or ϵ-aminohexanoic acid. In some embodiments, the peptide spacer is 1-5, or 1-2 amino acids in length.

In some embodiments, the spacer comprises a non-peptide spacer. This may be in addition to, or as an alternative to, a peptide spacer. The non-peptide spacer may comprises a straight chain, preferably wherein the non-peptide spacer comprises the group —(CH₂)ₐ—, wherein a is 1-20, preferably 1-15, 1-10, 1-5, or 2-4.

In preferred embodiments, the material compri sea one or both of the following structures:

In some embodiments, the substrate is a wound dressing. The wound dressing material is preferably a non-colloidal porous dressing material. The term “non-colloidal porous dressing material” is used herein to refer to any non-colloidal porous materia [symbol] topically applied to cover, dress, or protect a wound. Examples of such materials include sheets, pads, sponges, foams, films, gauzes, mesh, granules, and beads. Non-colloidal porous dressing material includes material that is suitable for topical application to a wound, but not suitable for injection into the body. In particular, the granules or beads are too large to pass through the lung capillary bed. At least a majority of the granules or beads have a maximum dimension that is greater than 6 μm. The dressing material is preferably oxidised cellulose, or any other dressing material that has carboxyl groups available for conjugation. The wound dressing may be a fabric.

Preferably, the wound dressing is sterile. The wound dressing may be provided as a sterile wound dressing ready for administration to a wound. The wound dressing may be packaged with instructions for application of the wound dressing to a wound.

The invention may provide a method of reducing or controlling bleeding, comprising administering the wound dressing to a wound.

In some embodiments, the substrate is a surgical fastener. The term “surgical fastener” refers to a means or agent for mechanically joining tissue, which is applied by piercing or puncturing tissue. Examples of surgical fasteners include sutures, staples and pins. A surgical fastener to which fibrinogen binding peptides have been covalently immobilised may be particularly advantageous for promoting clotting in holes created during its application. For example, it may prevent or reduce suture hole bleeding. Surgical fasteners may be used to attach materials such as wound dressings, to a patient.

The invention may provide a method of joining tissue comprising applying the surgical fastener to a patient.

Preferably, the peptides covalently immobilised to the substrate (or for covalent immobilisation to the substrate) e.g. fibrinogen-binding peptides, are each 4-60, preferably 4-30, more preferably 4-10, amino acid residues in length. In other embodiments, each peptide may be at least 3, 4, 5, 6, 7, 8, 9, 10, or 11 amino acid residues in length. It is preferred that each peptide is no longer than 60 amino acid residues in length, more preferably no longer than 30 amino acid residues in length.

The term “peptide” as used herein also incorporates peptide analogues. Several peptide analogues are known to the skilled person. In the context of a fibrinogen binding peptide, any suitable analogue may be used provided fibrinogen is able to bind the fi [symbol] binding peptide.

Examples of suitable fibrinogen binding peptides and how they may be identified are provided in WO 2005/035002, WO 2007/015107 and WO 2008/065388.

Preferably each fibrinogen-binding peptide is a synthetic peptide.

Preferably each fibrinogen binding peptide binds to fibrinogen with a dissociation constant (K_{D}) of between 10⁻⁹ to 10⁻⁶ M, for example around 10, 20, 30, 40, 50, 60, 70, 80, 90, 100, 110, 120, 130, 140, 150, 200, 250, 300, 350, 400, or more nM. A K_{D} of around 100 nM is preferred. The dissociation constant can be measured at equilibrium. For example, radio-labelled fibrinogen of known concentration can be incubated with microspheres to which the fibrinogen binding moiety has been cross-linked. Typically 5 μM peptide is cross-linked to 1 gm microspheres, or 15-40 μmoles of peptide is cross-linked to 1 gm of microspheres. The peptide-linked microspheres are diluted to 0.5 mg/ml, and incubated in isotonic buffer at pH 7.4 (for example 0.01 M Hepes buffer containing 0.15 M NaCl) with radio labelled fibrinogen at concentrations of between 0.05 and 0.5 mg/ml for up to 1 hr at 20° C. The fibrinogen bound to the fibrinogen binding moiety on the microspheres can be separated from the free fibrinogen by centrifugation and the amount of free and bound fibrinogen measured. The dissociation constant can then be calculated by Scatchard analysis by plotting concentration of bound fibrinogen against the ratio of the concentrations of bound: free fibrinogen, where the slope of the curve represents K_{D}.

A molecule of fibrinogen consists of three pairs of non-identical polypeptide chains, Aα, Bβ and γ, linked together by disulfide bonds. Fibrinogen chains are folded into three distinct structural regions, two distal D regions linked to one central E region. Each D region contains polymerization ‘a’ and ‘b’ holes located in the C terminus of the γ and Bβ chains, respectively. Thrombin catalyses the removal of short peptides, fibrinopeptides A (FpA) and B (FpB), from the amino-terminus of the Aα and Bβ chains of fibrinogen in the central E region, respectively, exposing two polymerisation sites: “knob A”, with amino-terminal sequence Gly-Pro-Arg-; and “knob B”, with amino-terminal sequence Gly-His-Arg-. The newly exposed polymerization knobs of one fibrin monomer interact with corresponding holes of another fibrin monomer through ‘A-a’ and ‘B-b’ knob-hole interactions, resulting in the assembly of fibrin monomers into half-staggered, double-stranded protofibrils.

In preferred embodiments of the invention, each fibrinogen binding peptide comprises the sequence Gly-(Pro, His)-Arg-Xaa (SEC) ID NO: 1) where Xaa is any amino acid and Pro/His means that either proline or histidine is present at that position. Preferably t [symbol] at an amino terminal end of the peptide. For example, the peptide may comprise the sequence NH₂-Gly-(Pro,His)-Arg-Xaa (SEQ ID NO: 1). The peptide may be attached to the substrate via its carboxy-terminal end.

However, in some embodiments, the amino acid sequence may be at a carboxy-terminal end of the peptide. The peptide may be attached to the substrate via its amino-terminal end. For example, at least one fibrinogen-binding peptide that binds preferentially to hole ‘a’ over hole ‘b’ of fibrinogen, such as a peptide comprising sequence APFPRPG (SEC) ID NO: 2), may be attached via its amino-terminal end to the carrier or to the thread. If the fibrinogen-binding peptide is attached via its amino-terminal end, the carboxy-terminal end of the peptide may comprise an amide group. The presence of an amide group, rather than a carboxyl group (or a negatively charged carboxylate ion), at the exposed carboxy-terminal end of the peptide may help to optimise binding of the fibrinogen-binding peptide to fibrinogen.

In some embodiments of the invention, at least some of the fibrinogen binding peptides comprise an amino acid sequence Gly-Pro-Arg-Xaa (SEQ ID NO: 3) wherein Xaa is any amino acid. Preferably, Xaa is any amino acid other than Val, and is preferably Pro, Sar, or Leu.

In some embodiments, at least some of the fibrinogen binding peptides comprise an amino acid sequence Gly-His-Arg-Xaa (SEQ ID NO: 4), wherein Xaa is any amino acid other than Pro.

According to some embodiments of the invention, the fibrinogen-binding peptides bind preferentially to hole ‘a’ of fibrinogen over hole ‘b’ of fibrinogen. Examples of sequences of suitable fibrinogen-binding peptides that bind preferentially to hole ‘a’ over hole ‘b’ of fibrinogen include: GPR-; GPRP-(SEQ ID NO: 5); GPRV-(SEQ ID NO: 6); GPRPFPA-(SEQ ID NO: 7); GPRVVAA-(SEQ ID NO: 8); GPRPWER-(SEQ ID NO: 9); GPRPAA-(SEQ ID NO: 10) ; GPRPPEC-(SEQ ID NO: 11); GPRPPER-(SEQ ID NO: 12); GPSPAA-(SEQ ID NO: 13).

According to some embodiments, the fibrinogen-binding peptides bind preferentially to hole of fibrinogen over hole ‘a’ of fibrinogen. Examples of sequences of fibrinogen-binding peptides that bind preferentially to hole ‘b’ over hole ‘a’ of fibrinogen include; GHR-, GHRP-(SEQ ID NO: 14), GHRPY-(SEG ID NO: 15), GHRPL-(SEQ ID NO: 16), GHRPYamide-(SEQ ID NO: 17).

The material may comprise fibrinogen-binding peptides of different sequenc [symbol] in some embodiments the material may comprise fibrinogen-binding peptides that have different selectivity of binding to hole ‘a’ over hole ‘b’ of fibrinogen.

Preferably, the fibrinogen-binding peptides do not comprise fibrinogen. Preferably, fibrinogen is not immobilised to the substrate. Preferably the material is not formed by immobilising fibrinogen to the substrate.

In a preferred arrangement, a fibrinogen molecule can bind at least two of the fibrinogen binding peptides. Consequently, if there is a plurality of fibrinogen binding peptides immobilised to the substrate, fibrinogen molecules may become non-covalently cross-linked. So, the fibrinogen binding peptides, may comprise one or more sequences that can bind to two distinct regions of fibrinogen, simultaneously. For example, fibrinogen comprises two terminal domains (D-domains), each of which may bind to a fibrinogen-binding peptide.

In a preferred embodiments, the material may comprise one or more of the following structures:

The invention may provide a method of controlling bleeding, comprising administering a haemiostatic material of the invention, to a wound.

In some embodiments, the substrate (preferably an oxidised cellulose substrate) is covalently immobilised to a plurality of fibrinogen binding peptides, in which the fibrinogen binding peptides are part of a haemostatic agent which has been covalently immobilised to the substrate.

For example, the haemostatic agent may comprise a plurality of carriers and [symbol] fibrinogen-binding peptides immobilised to each carrier. A plurality of the carriers may thus be covalently immobilised to the substrate and a plurality of fibrinogen binding peptides may be immobilised to each carrier.

In a preferred embodiment, the carriers are soluble carriers. For example, the carriers may be soluble in blood plasma. The carriers should be suitable for administration to a bleeding wound site, The carriers may comprise a polymer, for example a protein, a polysaccharide, or a synthetic biocompatible polymer, such as polyethylene glycol, or a combination of any of these. Albumin is a preferred protein carrier, The soluble carrier or haemostatic.: agent may have a solubility of at least 10 mg per ml of solvent, for example 10-1000 mg/ml, 33-1000 mg/ml, or 33-100 mg/ml

The carriers may comprise reactive groups which permit attachment of the fibrinogen-binding peptides. For example, the carriers may comprise thiol moieties or amine moieties on their surface. If the carriers are proteinaceous, the thiol or amine moieties may be provided by side chains of amino acids, for example cysteine or lysine. Alternatively, reactive groups may be added to the carrier. This is particularly advantageous if the carrier is formed from protein, such as albumin. For example, the carrier may be thiolated using a reagent such as 2-iminothiolane (2-IT) which is able to react with primary amine groups on the carrier. Alternatively cystamine may be coupled to carboxyl groups on the carrier in the presence of 1-Ethyl-3-(3-dimethylaminopropyl) carbodiimide hydrochloride (EDC) and N-hydroxysuccinimide (NHS), followed by reductive cleavage of the introduced disulphide bond.

In preferred embodiments, the fibrinogen-binding peptides are covalently immobilised to the carrier via a spacer. A preferred spacer is a non-peptide spacer, for example comprising a hydrophilic polymer such as polyethylene glycol (PEG). In a preferred embodiment, a plurality of peptide conjugates, each comprising a fibrinogen-binding peptide linked to a thiol-reactive group (for example, a maleimide group) by a PEG spacer are reacted with a thiolated carrier (for example prepared using 2-IT or cystamine as described above). Suitable non-peptide spacers are described in WO 2013/114132.

The haemostatic agent may comprise a peptide conjugate. A suitable carrier may thus comprise one or more amino acid residues, for example a single amino acid residue, such as a lysine amino acid residue, An advantage of conjugates comprising carriers that comprise one or more amino acid residues is that they can readily be made using solid phase peptide synthesis methods. In addition, they may be readily produced [symbol] immunogenic agents and may be resistant to sterilising radiation.

Each fibrinogen-binding peptide of the peptide conjugate may, independently, be attached at its carboxy-terminal end (optionally via a linker), or at its amino-terminal end (optionally via a linker), to the carrier.

In one example, the peptide conjugate may have the following general formula:

FBP-(linker)-X-(linker)-FBP

where:
FBP is a fibrinogen-binding peptide;
-(linker)- is an optional linker, preferably a non-peptide linker;
X is an amino acid, preferably a multifunctional amino acid, most preferably a tri-functional amino acid residue, such as lysine, ornithine, or arginine.

In a preferred embodiment, the peptide conjugate is a deridnmer. The dendrimer may comprise a branched core and a plurality of fibrinogen-binding peptides separately covalently attached to the branched core. The branched core may comprise one or more multifunctional amino acids. Each multifunctional amino acid, or a plurality of multifunctional amino acids, may have one or more fibrinogen binding peptides covalently attached to it.

The branched core may comprise: i) from two to ten multi-functional amino acid residues, wherein each fibrinogen-binding peptide is separately covalently attached to a multi-functional amino acid residue of the branched core; ii) a plurality of multi-functional amino acid residues, wherein one or more fibrinogen-binding peptides are separately covalently attached to each of at least two adjacent multi-functional amino acid residues of the branched core; iii) a plurality of multi-functional amino acid residues, wherein two or more fibrinogen-binding peptides are separately covalently attached to at least one of the multi-functional amino acid residues of the branched core; iv) a plurality of multi-functional amino acid residues, wherein two or more multi-functional amino acid residues are covalently linked through a side chain of an adjacent multi-functional amino acid residue; or v) a single multi-functional amino acid residue, and a fibrinogen-binding peptide is separately covalently attached to each functional group of the multi-functional amino acid residue.

The multi-functional amino acid residues may comprise tri- or tetra-functiona [symbol] residues, or tri- and tetra-functional amino acid residues, or the single multi-functional amino acid residue is a tri- or tetra-functional amino acid residue.

Each fibrinogen-binding peptide may have a different point of attachment to the branched core, so the fibrinogen-binding peptides are referred to herein as being “separately covalently attached” to the branched core.

The branched core comprises any suitable amino acid sequence. The branched core may comprise up to ten multi-functional amino acid residues, for example two to ten, or two to six multi-functional amino acid residues.

The branched core may comprise a plurality of consecutive multi-functional amino acid residues. The branched core may comprise up to ten consecutive multi-functional amino acid residues.

The term “tri-functional amino acid” is used herein to refer to any organic compound with a first functional group that is an amine (—NH₂), a second functional group that is a carboxylic acid (—COOH), and a third functional group. The term “tetra-functional amino acid” is used herein to refer to any organic compound with a first functional group that is an amine (—NH₂), a second functional group that is a carboxylic acid (—COOH), a third functional group, and a fourth functional group. The third and fourth functional group may be any functional group that is capable of reaction with a carboxy-terminal end of a fibrinogen-binding peptide, or with a functional group of a linker attached to the carboxy-terminal end of a fibrinogen-binding peptide.

Multifunctional amino acids may comprise a central carbon atom (α- or 2-) bearing an amino group, a carboxyl group, and a side chain bearing a further functional group (thereby providing a tri-functional amino acid), or a further two functional groups (thereby providing a tetra-functional amino acid.

The, or each, multi-functional amino acid residue may be a residue of a proteinogenic or non-proteinogenic multi-functional amino acid, or a residue of a natural or unnatural multi-functional amino acid.

Proteinogenic tri-functional amino acids possess a central carbon atom (α- or 2-) bearing an amino group, a carboxyl group, a side chain and an a-hydrogen levo conformation.

Examples of suitable tri-functional proteinogenic amino acids include L-lysine, L-arginine, L-aspartic acid, L-glutamic acid, L-asparagine, L-glutamine, and L-cysteine.

Examples of suitable tri-functional non-proteinagenic amino acid residues in [symbol] beta-Lysine, L-ornithine, D-ornithine, and D-arginine residues.

Thus, examples of suitable tri-functional amino acid residues for use in a peptide dendrimer of the invention include lysine, ornithine, arginine, aspartic acid, glutamic acid, asparagine, glutamine, and cysteine residues, such as L-lysine, D-lysine, beta-Lysine, L-ornithine, D-ornithine, L-arginine, D-arginine, L-aspartic acid, D-aspartic acid, L-glutamic acid, D-glutamic acid, L-asparagine, D-asparagine, L-glutamine, D-glutamine, L-cysteine, and D-cysteine residues.

Examples of suitable multi-functional unnatural amino acids suitable for use in a peptide dendnimer of the invention include Citrulline, 2,4-diaminoisobutyric acid, 2,2′-diaminopimelic acid, 2,3-diaminopropionic acid, and cis-4-amino-L-proline. Multi-functional unnatural amino acids are available from Sigma-Aldrich.

In some embodiments, the branched core may comprise a homopolymeric multi-functional amino acid sequence, for example a poly-lysine, poly-arginine, or poly-ornithine sequence, such as a branched core comprising from two to ten, or from two to six, consecutive lysine, arginine, or ornithine residues. In other embodiments, the branched core may comprise different multi-functional amino acid residues, for example one or more lysine residues, one or more arginine residues, and/or one or more ornithine residues.

In other embodiments, the branched core may comprise a plurality of multi-functional amino acid residues, and one or more other amino acid residues.

Where the branched core comprises a plurality of multi-functional amino acid residues, adjacent multi-functional amino acid residues may be linked together by amino acid side chain links, by peptide bonds, or some adjacent multi-functional amino acid residues may be linked together by side chain links and others by peptide bonds.

In further embodiments, the branched core may comprise two or more multi-functional amino acid residues, and at least one fibrinogen-binding peptide is separately attached to each of two or more of the multi-functional amino acid residues, and two or more fibrinogen-binding peptides are separately attached to at least one of the multi-functional amino acid residues of the branched core.

According to other embodiments, two fibrinogen-binding peptides are separately attached to a terminal multi-functional amino acid residue of the branched core.

Examples of structures of peptide dendrimers include peptide dendrimers in [symbol]
the branched core comprises a first tri-functional amino acid residue to which two fibrinogen-binding peptides are attached, and a second tri-functional amino acid residue to which one fibrinogen-binding peptide is attached;
the branched core comprises a first tri-functional amino acid residue to which two fibrinogen-binding peptides are attached, and a second tri-functional amino acid residue to which two fibrinogen-binding peptides are attached;
the branched core comprises a first tri-functional amino acid residue to which two fibrinogen-binding peptides are attached, a second tri-functional amino acid residue to which one fibrinogen-binding peptide is attached, and a third tri-functional amino acid residue to which one fibrinogen-binding peptide is attached; or
the branched core comprises a first tri-functional amino acid residue to which two fibrinogen-binding peptides are attached, a second tri-functional amino acid residue to which one fibrinogen-binding peptide is attached, a third tri-functional amino acid residue to which one fibrinogen-binding peptide is attached, and a fourth tri functional amino acid residue to which one fibrinogen-binding peptide is attached.

The peptide dendrimer may comprise the following general formula (I):

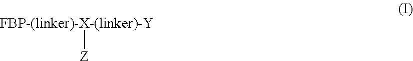

where:
FBP is a fibrinogen-binding peptide;
-(linker)- is an optional linker, preferably a non-peptide linker;
X is a tri-functional amino acid residue, preferably lysine, ornithine, or arginine;
Y is -FBP, or —NH₂;
Z is -(linker)-FBP when Y is -FBP, or —[—Xₙ-(linker)-FBP]ₐ-(linker)-FBP when Y is —NH₂;

where:
X is a tri-functional amino acid residue, preferably lysine, L-ornithine, or arginine; and
a is 1-10, preferably 1-3.

For example, when Y is NH₂, Z is —[—Xₙ-(linker)-FBP]ₐ-(linker)-FBP, the struct [symbol] dendrimer is as follows:
where a is 1:

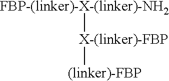

or, where a is 2:

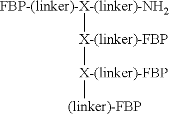

or, where a is 3:

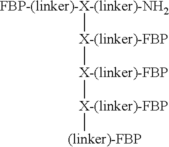

Alternatively, Z is —[—Xₙ-(linker)-FBP]ₐ(linker)-FBP when Y is -FBP;

where:
Xₙ is a tri-functional amino acid residue, preferably lysine, L-ornithine, or arginine; and
a is 1-10, preferably 1-3.

For example, when Y is -FBP, Z is —[—Xₙ-(linker)-FBP]ₐ-(linker)FBP and a is 1, the structure of the dendrimer is as follows:

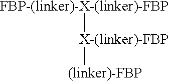

The peptide dendrimer may comprise the following general formula (H):

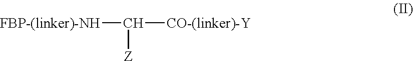

where:
FBP is a fibrinogen-binding peptide;
-(linker)- is an optional linker, preferably comprising —NH(CH₂)₅CO—;
Y is -FBP, or —NH₂;
Z is:
-R-(linker)-FBP, when Y is -FBP, or

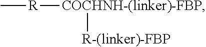

when Y is —NH₂; or

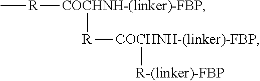

when Y is —NH₂; or

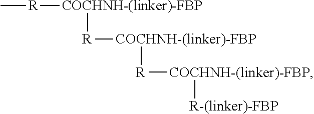

when Y is —NH₂;

where R is —(CH₂)₄NH—, —(CH₂)₃NH—, or —(CH₂)₃NHCNHNH—.

Consequently, in one embodiment, Z may be:

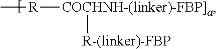

when Y is —NH₂;

where R is —(CH₂)4NH—, —(CH₂)₃NH—, or —(CH₁₂)₃NHCNHNH—;

where a is 1-3.

Alternatively, a may be 4-10, or it may be 1-10.

In another embodiment, Z is:

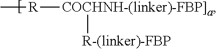

when Y is -FBP;

where R is —(CH₂)₄NH—, —(CH₂)₃NH—, or —(CH₂)₃NHCNHNH—;

where a is 1-10, preferably 1-3.

For example, Z is:

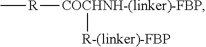

when Y is -FBP and a is 1.

The peptide dendrimer may comprise the following general formula (III):

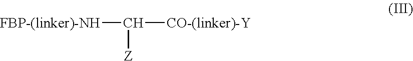

where:
FBP is a fibrinogen-binding peptide;
-(linker)- is an optional linker, preferably comprising —NH(CH₂)₅CO—;
Y is -FBP, or —NH₂;
Z is:
—(CH₂)₄NH-(linker)-FBP, when is -FBP; or

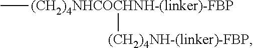

when Y is —NH₂; or

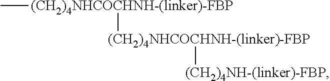

when Y is —NH₂; or

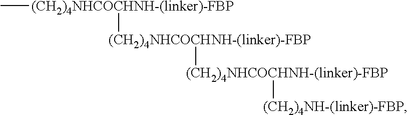

when Y is —NH₂,

Consequently, in one embodiment, Z may be:

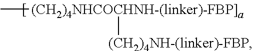

when Y is —NH2;

where a is 1-3.

Alternatively a is 4-10, or it may be 1-10.

In another embodiment, Z is:

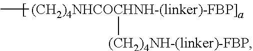

when Y is -FBP;

where a is 1-10, preferably 1-3.

For example, Z is;

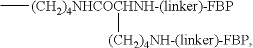

when Y is -FBF and a is 1.

One or more, or each, fibrinogen-binding peptide may be covalently attached by a non-peptide linker. The linker may be any .suitable linker that does not interfere with binding of fibrinogen to fibrinogen-binding peptides. The linker may comprise a flexible, straight-chain linker, suitably a straight-chain alkyl group. Such linkers may thus allow the fibrinogen-binding peptides of the peptide dendrimer to extend away from each other. For example, the linker may comprise a —NH(CH₂)ₙCO— group, where n is any number, suitably 1-10, for example 5. A linker comprising a —NH(CH₂)₅CO— group may be formed by use of ϵ-amino acid 6-aminohexanoic acid (ϵAhx).

A particular advantage of peptide conjugates, such as peptide dendrimers, is that they can readily be sterilised, for example by exposure to irradiation, suitably gamma irradiation, without significant loss of the ability of the peptide dendrimer, or compositio [symbol] with fibrinogen.

According to the invention, there is provided a method of sterilising a haemostatic material comprising exposing the material to gamma irradiation, preferably up to 30 kGy, wherein the haemostatic material comprises a substrate (preferably an oxidised cellulose substrate) and a plurality of fibrinogen binding peptides immobilised to the substrate. Preferably, the fibrinogen binding peptides are provided by peptide conjugates, such as peptide dendrimers.

In theory there is no upper limit to the number of fibrinogen-binding peptides per carrier. However, in practice, for any particular structure, the number of fibrinogen-binding peptides can be varied and tested to determine the optimum number for the desired fibrinogen polymerisation properties, for example, for the speed fibrinogen polymerisation or for the density of the hydrogel produced by polymerisation with fibrinogen. The optimum number is likely to depend on many factors, such as the nature of the carrier, and the number of reactive groups on each carrier for attaching the fibrinogen-binding peptides. However, it is preferred that on average there are up to 100 fibrinogen-binding peptides per carrier molecule. Preferably, on average there are at least three, preferably at least, five fibrinogen-binding peptides per carrier molecule. A preferred range is 10-20 fibrinogen-binding peptides per carrier molecule. Peptide conjugates, such as peptide dendimers may comprise a total of up to twenty fibrinogen-binding peptides per dendrimer, for example up to ten fibrinogen-binding peptides per dendrimer, or up to five fibrinogen-binding peptides per dendrimer.

Each carriers may have fibrinogen binding peptides of different sequence attached to it. For a plurality of carriers, a first plurality may have a different fibrinogen binding peptide attached compared to a second plurality of carriers.

A haemostatic agent suitable for immobilisation to the substrate may comprise a peptide dendrimer, and a peptide conjugate comprising two or more fibrinogen-binding peptides. The peptide conjugate may comprise fibrinogen-binding peptides of the same sequence, or of different sequence. For example, the peptide conjugate may comprise only fibrinogen-binding peptides that bind preferentially to hole ‘a’ over hole ‘b’ of fibrinogen, or only fibrinogen-binding peptides that bind preferentially to hole ‘b’ over hole ‘a’ of fibrinogen, or one or more fibrinogen-binding peptides that bind preferentially to hole ‘a’ over hole ‘b’ of fibrinogen and one or more fibrinogen-binding peptides that bind preferentially to hole ‘b’ over hole ‘a’ of fibrinogen. In some embodiments, the peptide conjugate may be a peptide dendrimer. The fibrinogen-binding peptides of the peptide dendrimer may bi [symbol] preferentially to hole ‘a’ of fibrinogen over hole ‘b’ of fibrinogen, and the fibrinogen-binding peptides of the peptide conjugate may bind preferentially to hole ‘b’ of fibrinogen over hole ‘a’ of fibrinogen, Such compositions have been found to have synergistic effects in that they are able to polymerise fibrinogen more rapidly than either the peptide dendrimer or the peptide conjugate alone. The mechanism of this synergistic effect is not fully understood, but without being bound by theory, it is believed that it may occur because the composition provides more ‘A’ and ‘B’ fibrinogen polymerisation sites.

Alternatively, the fibrinogen-binding peptides of the peptide dendrimer may bind preferentially to hole ‘b’ of fibrinogen over hole ‘a’ of fibrinogen, and the fibrinogen-binding peptides of the peptide conjugate bind preferentially to hole ‘a’ of fibrinogen over hole ‘b’ of fibrinogen

Embodiments of the invention are now described by way of example only, with reference to the accompanying drawings, in which:

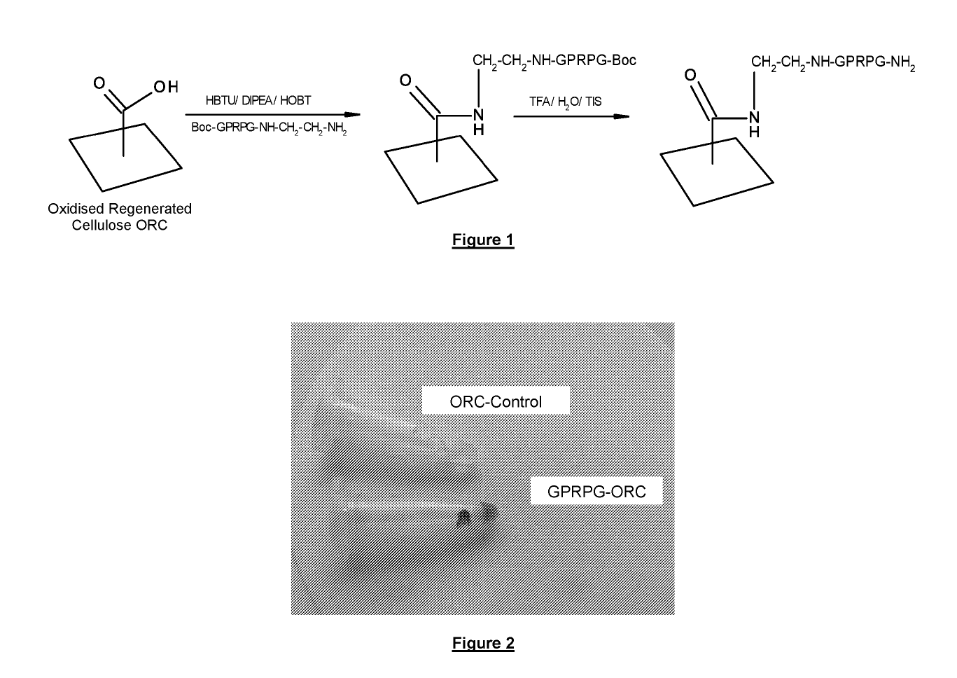

FIG. 1 shows a scheme for synthesising a haemostatic dressing of the invention;

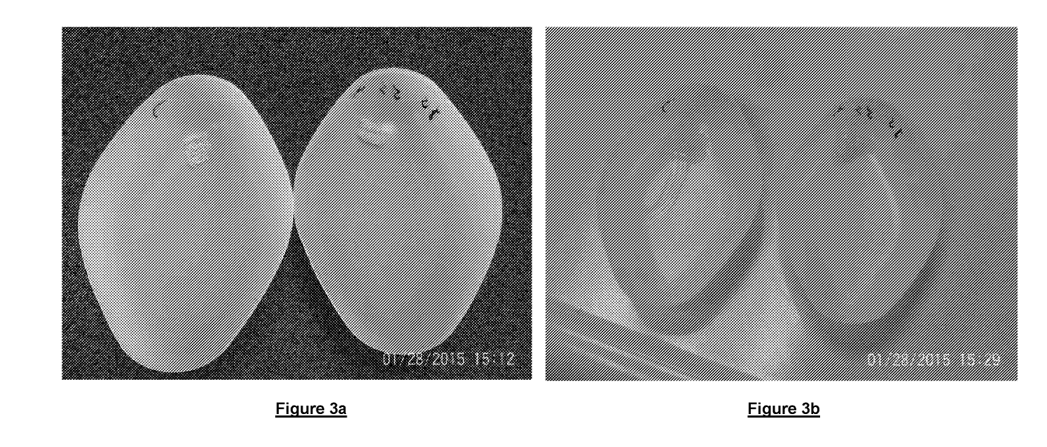

FIG. 2 shows the results of the Kaiser test (Ninhydrin test) used to monitor presence of fully deprotected fibrinogen peptide remaining bound on a dressing of the invention;

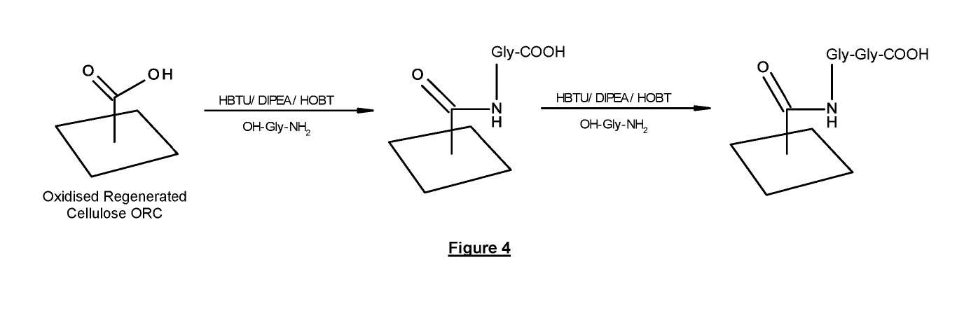

FIG. 3a shows samples of a control dressing and a haemostatic dressing of the invention;

FIG. 3b shows improved clotting activity of a dressing of the invention compared to a control;

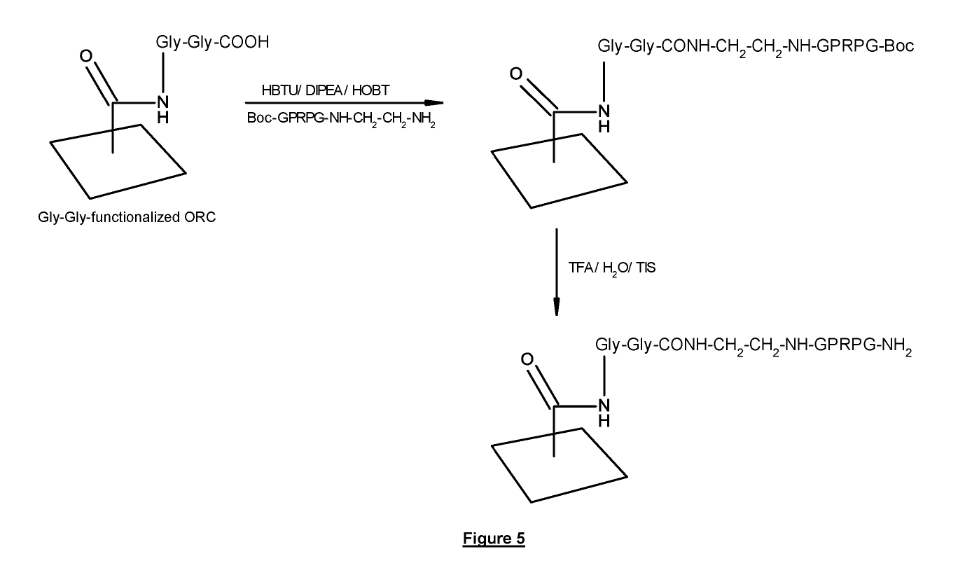

FIG. 4 shows a reaction scheme for modifying the surface of a dressing;

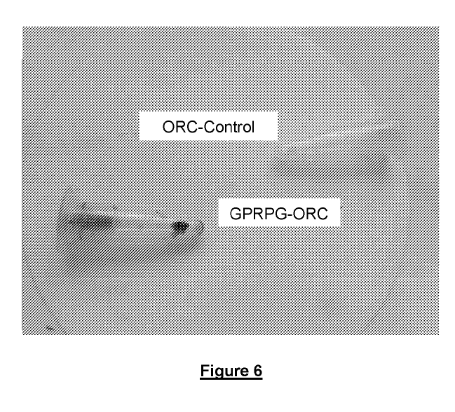

FIG. 5 shows a reaction scheme for synthesising, a haemostatic dressing of the invention;

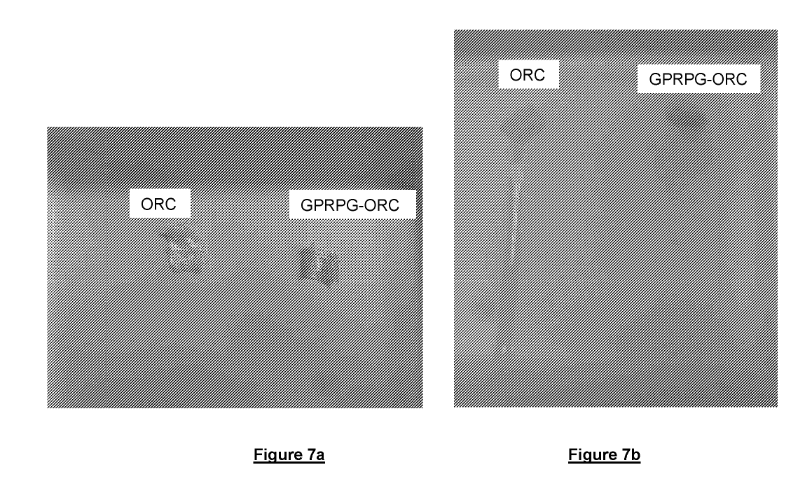

FIG. 6 shows the results of the Kaiser Test (Ninhydrin test) used to monitor presence of fully deprotected fibrinogen peptide remaining bound on a dressing of the invention;

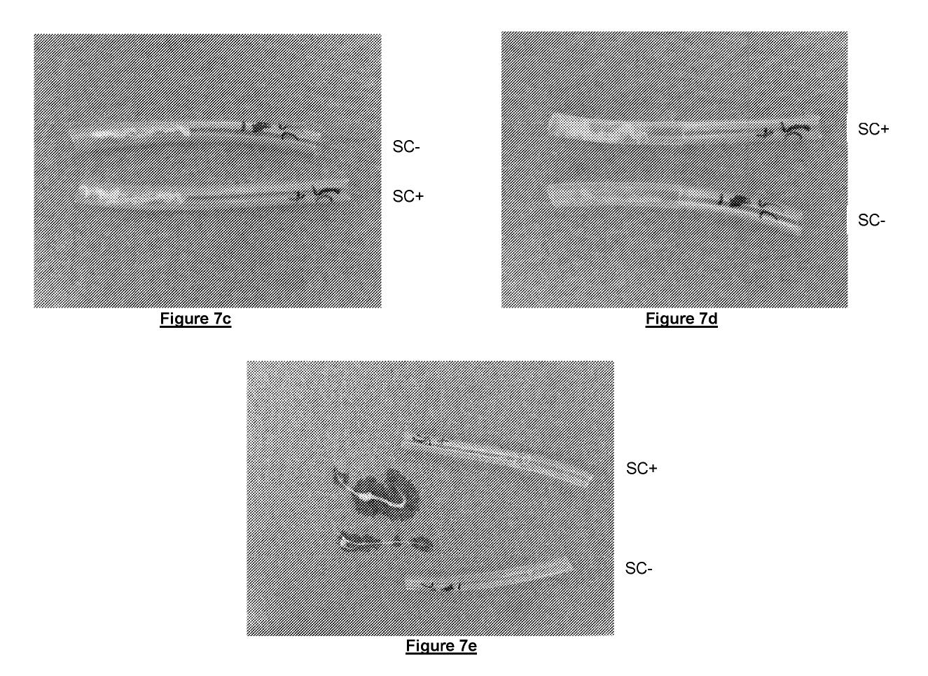

FIG. 7a shows samples of a control dressing and a haemostatic dressing of the invention;

FIG. 7b shows improved clotting activity of a dressing of the invention compared to a control;

FIG. 7c shows a haemostatic material of the invention and a control material placed into polypropylene tubes;

FIG. 7d shows polymerisation of human fibrinogen by a haemostatic material of the invention;

FIG. 7e shows a fibrinogen clot on a haemostatic material of the invention;

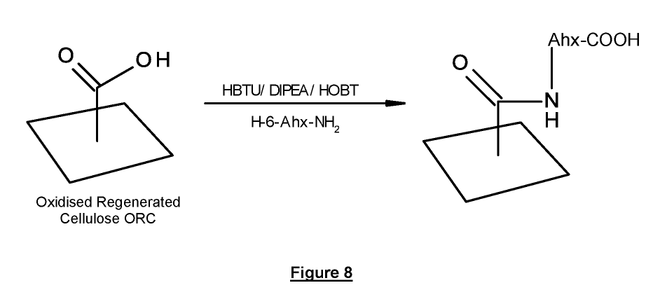

FIG. 8 shows a reaction scheme for modifying the surface of a dressing;

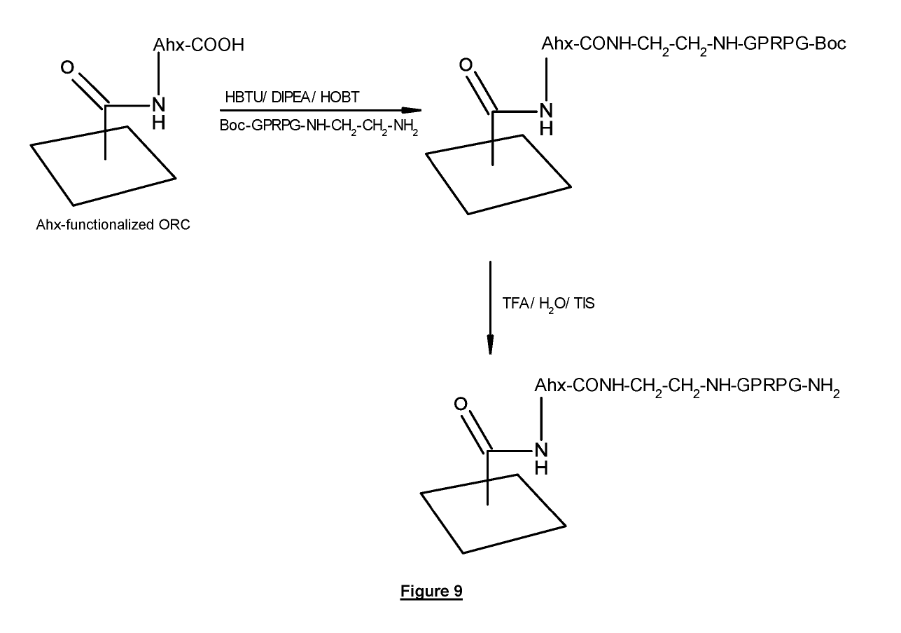

FIG. 9 shows a reaction scheme for synthesising a haemostatic dressing of the invention;

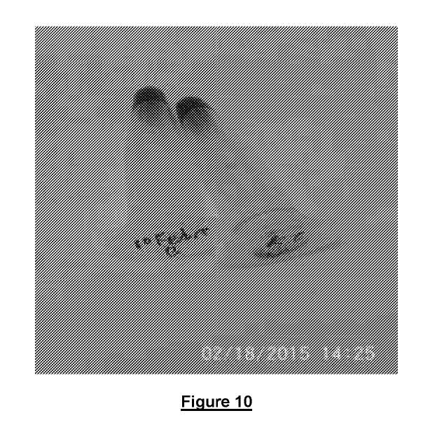

FIG. 10 shows the results of the Kaiser Test (Ninhydrin test) used to monitor presence of fully deprotected fibrinogen peptide remaining bound on a dressing of the invention;

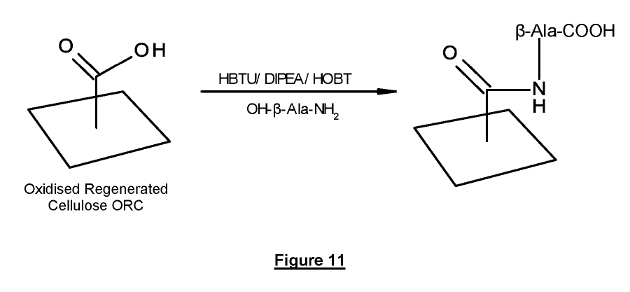

FIG. 11 shows a reaction scheme for modifying the surface of a dressing;

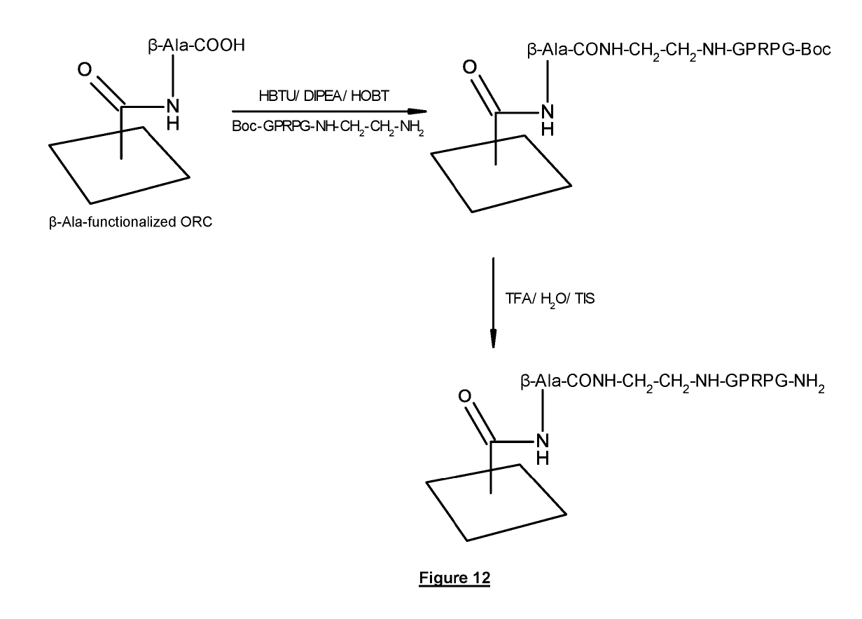

FIG. 12 shows a reaction scheme for synthesising a haemostatic dressing of the invention;

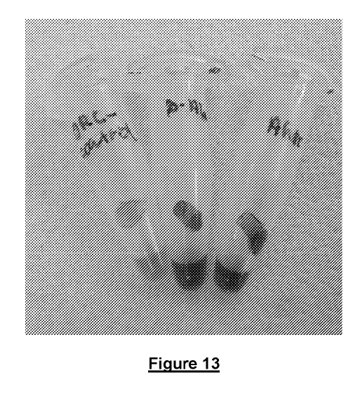

FIG. 13 shows the results of the Kaiser Test (Ninhydrin test) used to monitor presence of fully deprotected fibrinogen peptide remaining bound on a dressing of the invention;

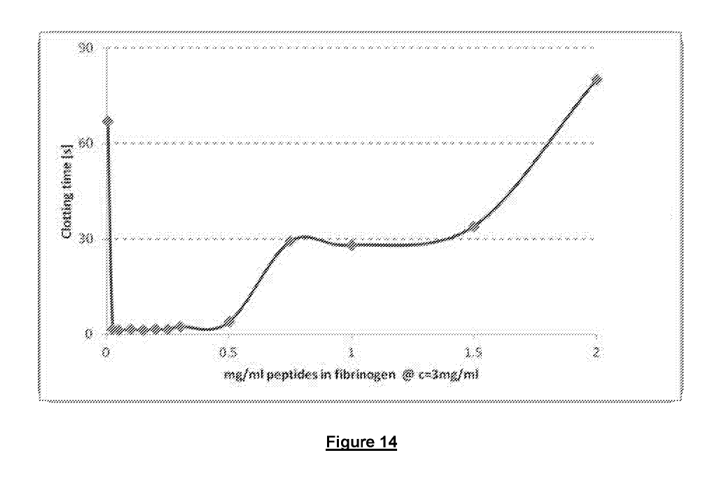

FIG. 14 shows the ability of a peptide dendrimer to polymerise fibrinogen at varying concentrations;

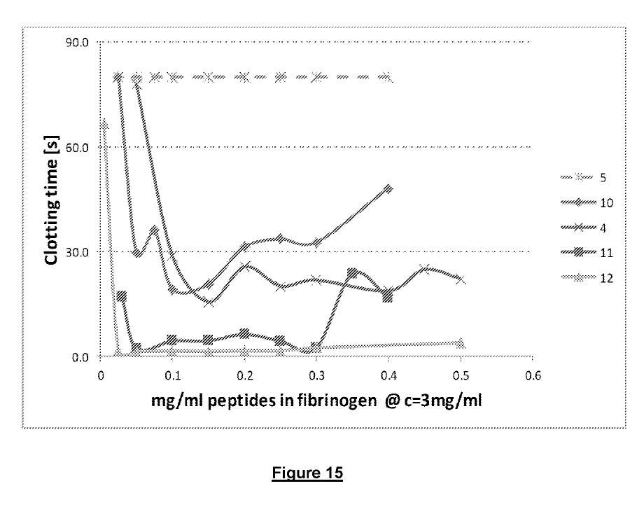

FIG. 15 shows the ability of several different peptide dendrimers to polymerise fibrinogen at varying concentrations. The numbering refers to the identity of the peptide dendrimer;

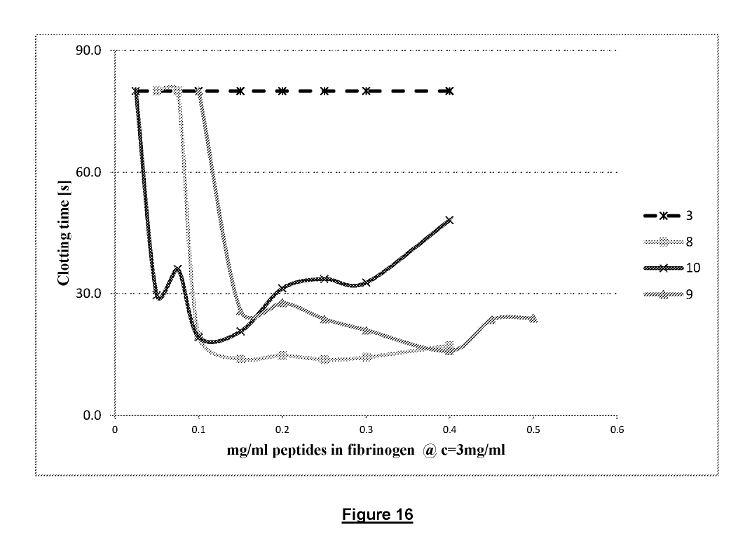

FIG. 16 shows the ability of several different peptide dendrimers to polymerise fibrinogen at varying concentrations. The numbering refers to the identity of the peptide dendrimer;

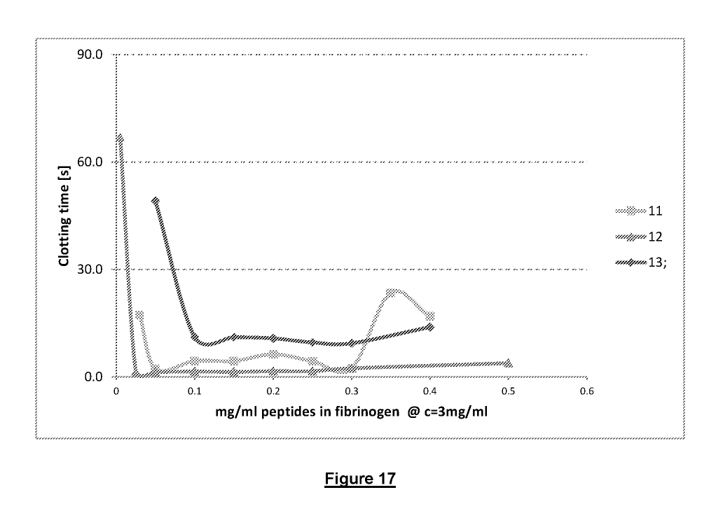

FIG. 17 shows the ability of several different peptide dendrimers to polymerise fibrinogen at varying concentrations. The numbering refers to the identity of the peptide dendrimer;

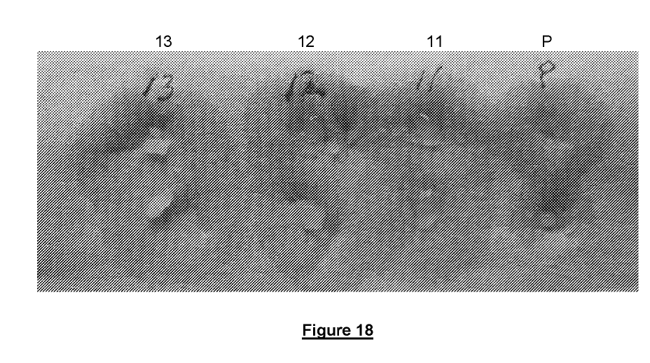

FIG. 18 shows a photograph of hydrogels formed by polymerisation of f [symbol] different peptide dendrimers;

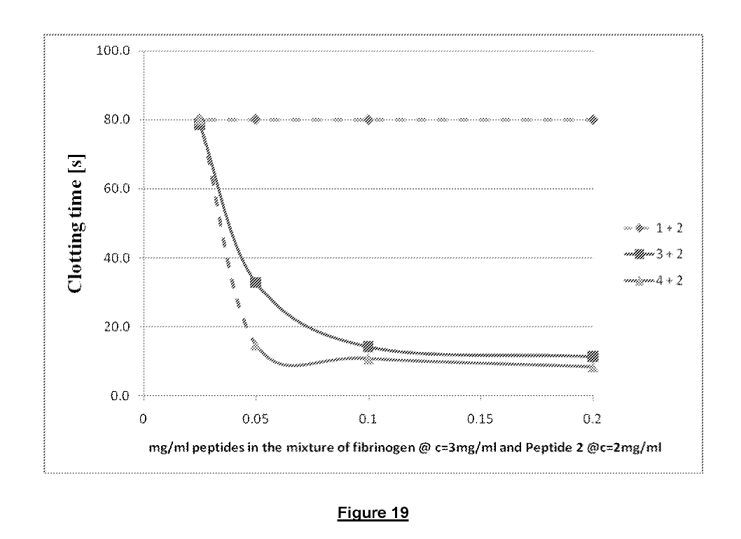

FIG. 19 shows the ability of different combinations of peptide dendrimers with peptide conjugates to polymerise fibrinogen at varying concentrations; and

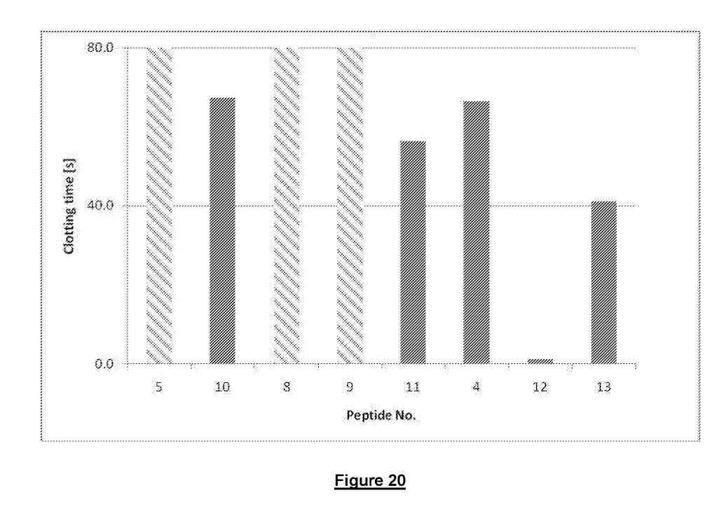

FIG. 20 shows the ability of several different peptide dendrimers to polymerise fibrinogen in human plasma.

### EXAMPLE 1

### One Step Coupling of Boc-GPR (Pbf) PG—NH—CH₂—CH₂—NH₂ (Boc-FBP-) on Oxidized Regenerated Cellulose Fabric

Boc-CPP (Pbf) PG—NH—CH₂—CH₂—NH₂ (Boc-FBP-) moieties were assembled from the C to N terminus exclusively by Fmoc-chemistry. During the last synthetic point of the synthesis, the moieties were fully protected (including a Pbf protection group on Arg), except for a free amino group on the C-termini, and. Protected moieties were purchased from Almac Ltd.

Commercially available Surgicel* Absorbable Hemostat (oxidized regenerated cellulose (ORC)) made by Ethicon Inc. of Johnson & Johnson Medical Limited was used as the substrate. Carboxylic acid content in Surgical was adopted from the literature (See EP 0659440). 50 grams of Surgicel Nu-Knit* cloth has 20% carboxylic acid content (0.22 moles of carboxylic acid).

ORC fabric used in the synthesis was pre-washed with 2×1 ml dichloromethane (DCM) (1 min) and dried at 33° C. After drying, the ORC fabric -50 mg (0.2 mmol—of carboxylic acid COOH) was immersed in a 1 ml dimethylformamide (DMF) solution and mixed with O-benzotriazole-N,N,N′,N′-tetramethyl-uronium-hexafluoro-phosphate (HBTU; 90 mg, 0.2 mmol). 1-hydroxy-1H-benzotriazole (HOBT; 30 mg, 0.2 mmol) then dressing was activated for 15 renins at room temperature. N,N-Diisopropylethylenediamine (0.4 mmol, 0.075 ml, d=0.798) (or N,N-Diisopropylethylamine DIPEA) was then added and resulting solution reacted for another 15 min. After this, 50 mg, 0.05 mmol of Boc-GPR (Pbf) PG—NH—CH₂—CH₂—NH₂ dissolved in DMF was added to the reaction mixture—2 ml in total. The coupling reaction was carried on at room temperature for 5 hours. The fabric was washed with DMF (3×1 ml), Methanol (MeOH) (3×1 ml) and with DMF (3×m1). The Boc-GPR (Pbf) PG—NH—CH₂—CH₂—NH₂ coupling step was repeated and incubated overnight at room temperature then washed with DMF (2×1 ml), MeOH (1×1 ml) and with DMF (2×1 ml). The ORC fabric was then washed with DMF (3×5 ml) and with DCM (3×5 ml). Removal of protecting groups with 95% TFA, 2.5% TIS, 2.5% water (3 ml) after the c [symbol] produced GPRPG—NH—CH₂—CH₂—NH—CO—ORC (“GPRPG-ORC”).

FIG. 1 summarises the reaction scheme and structures. FIG. 2 shows the results of a Kaiser Test (Ninhydrin test) tests to monitor the presence of fully deprotected peptide remaining bound on the fabric (ORC control (top); GPRPG-ORC (bottom)).

### EXAMPLE 2

### Functionality Test

Samples of GPRPG-FBP and ORC (control) were weighed out, treated with 100 μl of human plasma (Alpha Labs-Plasma Lot# A1162 Exp 2015-03) and incubated for 1.5 or 3 min at 33′C. Tested samples and controls were removed from the plasma and then were weighed to determine any difference. The test was repeated 3 times. The results in Table 1 show that the mass remaining on the GPRPG-ORC is significantly higher compared to control samples, indicating that fibrinogen binding peptides retain activity when conjugated to the fabric.

TABLE 1

| | | | Incubation time with |
|---|---|---|---|
| | Starting | End | 100 μl of human |
| Tested samples | mass (mg) | mass (mg) | plasma (min) |
| ORC (control) | 6 | 42 | 3 |
| GPRPG-ORC | 6 | 66 | 3 |
| ORC (control) | 5 | 21 | 1.5 |
| GPRPG-ORC | 5 | 45 | 1.5 |
| ORC (control) | 3 | 27 | 1.5 |
| GPRPG-ORC | 3 | 42 | 1.5 |

Samples of ORC (control) and GPRPG-ORC (6 mg each) were placed on weighing boat (See FIG. 3a—control (left); GPRPG-ORC (right)) and treated with 100 μl of human plasma then incubated for 3 min at 33° C. The resulting clots were placed (tilted) to 90° angle, and any run-off from the clot was observed. ORC (control) released fluid, but GPRPG-ORC did not (See FIG. 3b control (left); GPRPG-ORC (right)),

### EXAMPLE 3

### Preparation of Surface-Modified Oxidised Cellulose fabric with a Gly-Gly Spacer

Introduction of a Gly-Gly spacer into the oxidised cellulose fabric was accor [symbol] through base catalysed HBTU/HOBT amide bond formation. Fabric used in the synthesis was pre-washed with 2×5 ml dichloromethane (DCM) (1 min) and dried at 33° C. After drying, the fabric—285 mg (1.25 mmol—COOH concentration) was immersed in a 5 ml dimethylformamide (DMF) solution containing and mixed with O-benzotriazole-N,N,N′,N′-tetramethyl-uronium-hexafluoro-phosphate (HBTU; 597 mg, 1.156 mmol), 1-hydroxy-1H-benzotriazole (HOBT; 217 mg, 1.56 mmol) then the fabric was activated for 15 min at room temperature, N,N-Diisopropylethylenediamine (3.14 mmol, 0.505 ml, d=0.798) (or N,N-Diisopropylethylamine DIPEA) was then added and resulting solution reacted for another 15 min. After this, 24 mg, 0.31 mmol of Gly-OH dissolved in Dimethylsulfoxide (DMSO) was added to the reaction mixture.

The coupling reaction was carried out at room temperature for 140 min. Oxidised cellulose fabric was washed with DMF (3×5 ml), Methanol (MeOH) (3×5 ml) and with DMF (3×5 ml). The Gly-OH coupling step was repeated and incubated for 30 min at room temperature then washed with DMF (2×5 mi), MeOH (1×5 ml) and with DMF (2×5 ml).

FIG. 4 summarises the reaction scheme and structures.

### EXAMPLE 4

### Coupling of Boc-FBP to Gly-Gly-Functionalised Oxidised Cellulose Fabric

The coupling of the Boc-FBP to the Gly-Gly-functionalised fabric was accomplished by a base-catalysed synthesis approach. First, Gly-Gly-functionalised fabric was immersed in a DMF (5 ml) and mixed with HBTU (475 mg, 1.25 mmol), HOBT (159 mg, 1.25 mmol). After stirring at room temperature for 2 min, N,N-Diisopropylethylenediamine (0.406 ml, 2.5 mmol) (or DIPEA) was added and mixed for 2 min. 275 mg (0.31 mmol) of Boc-FBP peptide was dissolved in DMF (200 μl) and this was added to the reaction mixture. The coupling reaction was carried out overnight (17 hours) at room temperature. The dressing then was washed with DMF (3×5 ml) and with DCM (3×5 ml). Removal of protecting groups with 95% TFA, 2.5% TIS, 2.5% water (3 ml) after the coupling reaction produced GPRPG—NH—CH₂—CH₂—NH—CO-G-G-ORC (“GPRPG-G-G-ORC”).

The Kaiser test (Ninhydrin test) was used to monitor presence of fully deprotected peptide remaining bound on the fabric (See FIG. 6—ORC control (top); GPRPG-G-G-ORC (bottom)).

The Kaiser Test showed a strong positive result that was more intense comp [symbol] Example 1 (without Gly-Gly spacer).

### Example 5

### Functionality Test

The tilt test (described in Example 2) was repeated for GPRPG-G-G-ORC. ORC (control) and GPRPG-G-G-ORC (8 mg each) were each placed in a weighing boat and treated with 100 μl of human plasma than incubated for 1.5 min at 33° C. The resulting clots were placed (tilted) to 90° angle. The strength of the clot was observed. FIGS. 7a and 7b illustrate that GPRPG-G-G-ORC samples formed a stronger clot with plasma than ORC control (left); (GPRPG-G-G-ORC (right)).

Samples of GPRPG-G-G-FBP and ORC (control) were weighed out and treated with 150 μl of human plasma (Alpha Labs-Plasma Lot# A1174 Exp 2016-03) and incubated for 1.5 min at 33° C. Tested samples and controls were removed from the plasma and then were weighed to determine if any difference was observable. The test was repeated four times. The results in table 2 showed that the mass remaining on GPRPG-G-G-ORC was significantly higher compared to control samples, suggesting that fibrinogen-binding peptides retain activity when conjugated to the regenerated oxidized cellulose fabric.

TABLE 2

| | | | Incubation time with |
|---|---|---|---|
| | Starting | End | 150 μl of human |
| Tested samples | mass (mg) | mass (mg) | plasma (min) |
| ORC (control) | 8 | 65 | 1.5 |
| GPRPG-G-G-ORC | 8 | 89 | 1.5 |
| ORC (control) | 9 | 50 | 1.5 |
| GPRPG-G-G-ORC | 9 | 83 | 1.5 |
| ORC (control) | 6 | 43 | 1.5 |
| GPRPG-G-G-ORC | 7 | 50 | 1.5 |
| ORC-control | 10 | 78 | 1.5 |
| GPRPG-G-G-ORC | 10 | 85 | 1.5 |

FIG. 7c shows GPRPG-G-G-ORC (tube labelled SC+ (top)) and ORC (control) (tube labelled SC− (bottom)) fibres placed into separate polypropylene tubes. 150 μl of Human Plasma solution (Alpha Labs-Plasma Lot# A1162 Exp 2016-03) was added to each sample and the fibres were incubated at 37′C for 1.5 minutes. There is a cl [symbol] SC+ − shown in FIG. 7d.

Visual examination of threads removed from the polyethylene tubes was also undertaken. FIG. 7e shows that GPRPG-G-G-ORC fibre formed a clot with human fibrinogen. The GPRPG-G-G-ORC fibre removed from the container was thicker than the control sample.

### EXAMPLE 6

### Preparation of Surface-Modified Oxidised Cellulose Fabric with ϵ-Ahx Spacer

Introduction of 6-amino hexanoic acid (ϵ-Ahx) spacer into the oxidsed cellulose fabric was accomplished through base catalysed HBTU/NHOBT amide bond formation. The synthetic method employed was substantially the same as described above in Example 3 for modification of ORC fabric with Gly-Gly spacers.

After prewashing and drying steps. 114 mg of fabric was immersed in 2 ml of DMF solution and mixed with O-benzotriazole-N,N,N′,N′-tetramethyl-uronium-hexafluoro-phosphate (HBTU; 237 mg, 0.625 mmol), 1-hydroxy-1H-benzotriazole (HOST; 84 mg, 0.625 mmol) then the fabric was activated for 15 min at room temperature. N,N-Diisopropylethylenediamine (1.25 mmol, 0.200 ml, d=0.798) (or DIPEA) was then added and resulting solution reacted for another 15 min. After this, 16.4 mg, 0.125 mmol of ϵ-Ahx-OH dissolved in Dimethylsulfoxide (DMSO) was added to the reaction mixture, The coupling reaction was carried out at room temperature overnight.

The fabric was washed with DMF (3×3 ml), Methanol (MeOH) (3×ml) and with DMF (3×3 ml).

FIG. 8 summarises the reaction scheme and the structures.

### EXAMPLE 7

### Coupling of Boc-FBP to the Ahx-Functionalised Dressing

The coupling of the Boc-FBP to the Ahx-functionalised fabric was accomplished by a base catalysed synthesis approach as described in Example 4.

A summary of the reaction scheme, and the structures, is shown in FIG. 9.

Firstly, Ahx-functionalised dressing was immersed in a DMF (2 ml) and mixed with HBTU (190 mg, 0.5 mmol), HOBT (67.4 mg, 0.5 mmol). After stirring at room temperature for 2 min N,N-Diisopropylethylenediamine (0.180 ml, 1.1 mmol) (or IPEA) was added and mixed for 2 min, 110 mg (0.125 mmol) of Boc-FOP peptide was dissolved in DMF (200 μl) and this was added to the reaction mixture. The coupling reaction was carried out overnight (17 hours) at room temperature. The dressing then was washed with DMF (3×3 ml) and with DCM (3×3 ml). Removal of protecting groups with 95% TFA, 2.5% TIS, 2.5% water (3 ml) after the coupling reaction produced GPRPG-NH—CH₂—CH₂—NH——CO-Ahx-ORC (“GPRPG-Ahx-ORC”).

The Kaiser test (Ninhydrin test) was used to monitor presence of fully deprotected peptide remaining bound on the cellulose (See FIG. 10—GPRPG-Ahx-ORC (left); GPRPG-G-G-ORC (right)).

### EXAMPLE 8

### Preparation of Surface-Modified Oxidised Cellulose Fabric with β-Ala Spacer

Introduction of β-alanine (β-Ala) spacer into the oxidsed cellulose fabric was accomplished through base catalysed HBTU/HOBT amide bond formation. The synthetic method employed was substantially the same as described above in Example 3 for modification of ORC fabric with Gly-Gly spacers.

After prewashing and drying steps, 206 mg of fabric was immersed in 5 ml of DMF solution and mixed with O-benzotriazole-N,N,N′,N′-tetramethyl-uronium-hexafluoro-phosphate (HBTU; 442 mg, 1.165 mmol), 1-hydroxy-1H-benzotriazole (HOBT; 152 mg, 1.125 mmol) then the fabric was activated for 15 min at room temperature. N,N′-Diisopropylethylenediamine (2.468 mmol, 0.319 ml) (or N,N-Diisopropylethylamine-DIEPA) was then added and resulting solution reacted for another 15 min. After this, 20 mg, 0.226 mmol of β-Ata-OH dissolved in Dimethylsulfoxide (DMSO) was added to the reaction mixture. The coupling reaction was carried out at room temperature overnight.

The fabric was washed with DMF (3×5 ml), Methanol (MeOH) (3×5 ml) and with DMF (3×5 ml).

FIG. 11 summarises the reaction scheme and the structures.

### EXAMPLE 9

### Coupling of Boc-FBP to the β-Ala-Functionalised Dressing

The coupling of the Boc-FBP to the β-Ala-functionalised fabric was accomplished by a base catalysed synthesis approach as described in Example 4.

A summary of the reaction scheme, and the structures, is shown in FIG. 12.

Firstly, β-Ala-functionalised dressing was immersed in a DMF (5 ml) and mixed with HBTU (350 mg, 0.923 mmol), HOBT (124 mg, 0.918 mmol). After stirring at room temperature for 2 min N,N′-Diisopropylethylenediamine (0.247 ml, 1.911 mmol) (or N,N-Diisopropylethylamine DIEPA) was added and mixed for 2 min. 202 mg (0.231 mmol) of Boc-FBP peptide was dissolved in DMF (400 μl) and this was added to the reaction mixture. The coupling reaction was carried out overnight (17 hours) at room temperature. The dressing then was washed with DMF (3×5 ml) and with DCM (3×5 ml). Removal of protecting groups with 95% TFA, 2.5% TIS, 2.5% water (3 ml) after the coupling reaction produced GPRPG-NH—CH₂—CH₂—NH—CO-Ahx-ORC (“GPRPG-Ahx-ORC”).

The Kaiser Test was used to monitor the presence of fully deprotected peptide remaining bound on the cellulose. See FIG. 13 from left Surgical control (labelled as ORC-Control), GPRPG-β-Ala-ORC and GPRPG-Ahx-ORC.

### EXAMPLE 10

### Functionality Test

Samples of GPRPG-β-Ala-FBP, GPRPG-Ahx-ORC and ORC (control) were weighed out and treated with 100 μl of human plasma (Alpha Labs-Plasma Lot# A1174 Exp 2016-03) and incubated for 1.5 min at 33° C. Tested samples and controls were removed from the plasma and then were weighed to determine if any difference was observable. The test was repeated three times. The results in table 3 showed that the mass remaining on GPRPG-β-Ala-ORC, GPRPG-Ahx-ORC was significantly higher compared to control (Surgicel) samples, suggesting that fibrinogen-binding peptides retain activity when conjugated to the regenerated oxidised cellulose fabric.

TABLE 3

| | | | Incubation time with |
|---|---|---|---|
| | Starting | End | 100 μl of human |
| Tested samples | mass (mg) | mass (mg) | plasma (min) |
| ORC (control) | 4 | 34 | 1.5 |
| GPRPG-β-Ala-FBP | 4 | 64 | 1.5 |
| GPRPG-Ahx-ORC | 4 | 54 | 1.5 |
| ORC (control) | 4 | 36 | 1.5 |
| GPRPG-β-Ala-FBP | 4 | 60 | 1.5 |
| GPRPG-Ahx-ORC | 4 | 50 | 1.5 |
| ORC (control) | 4 | 32 | 1.5 |
| GPRPG-β-Ala-FBP | 4 | 59 | 1.5 |
| GPRPG-Ahx-ORC | 4 | 57 | 1.5 |

### EXAMPLE 11

### Synthesis of Peptide Dendrimers and Peptide Conjugates

Peptides were synthesised on Rink amide MBHA low loaded resin (Novabiochem, 0.36 mmol/g), by standard Fmoc peptide synthesis, using Fmoc protected amino acids (Novabiochem).

In general, single-coupling cycles were used throughout the synthesis and HBTU activation chemistry was employed (HBTU and PyBOP (from AGTC Bioproducts) were used as the coupling agents). However, at some positions coupling was less efficient than expected and double couplings were required.

The peptides were assembled using an automated peptide synthesiser and HBTU up to the branch points and by manual peptide synthesis using PyBOP for the peptide branches.

For automated synthesis a threefold excess of amino acid and HBTU was used for each coupling and a ninefold excess of N,N-Diisopropylethylamine (DIPEA, Sigma) in dimethylformamide (DMF, Sigma).

For manual synthesis a threefold excess of amino acid and PyBOP was used for each coupling and a ninefold excess of DIPEA in N-methylpyrollidinone (NMP, Sigma).

Deprotection (Fmoc group removal) of the growing peptide chain using 20% piperidine (Sigma) in DMF likewise may not always be efficient and require double deprotection.

Branches were made using Fmoc-Lys(Fmoc)-OH, Fmoc-Lys(Boc)-OH, or F [symbol] OH.

Final deprotection and cleavage of the peptide from the solid support was performed by treatment of the resin with 95% TFA (Sigma) containing triisopropylsilane (TIS, Sigma), water and anisole (Sigma) (1:1:1, 5%) for 2-3 hours.

The cleaved peptide was precipitated in cold diethyl ether (Sigma) pelleted by centrifugation and lyophilized. The pellet was re-dissolved in water (10-15 mL), filtered and purified via reverse phase HPLC using a C-18 column (Phenomenex at flow rate 20 ml/min) and an acetonitrile/water gradient containing 0.1% TFA. The purified product was lyophilized and analyzed by ESI-LC/MS and analytical HPLC and were demonstrated to be pure (>95%). Mass results all agreed with calculated values.

Peptide Dendrimers and Peptide Conjugates

The structures of peptide dendrimers and peptide conjugates synthesised using the methods described above are shown below.

The “NH₂—” group at the end of a peptide sequence denotes an amino group at the amino-terminal end of the sequence. The “-am” group at the end of a peptide sequence denotes an amide group at the carboxy-terminal end of the sequence.

Peptide Conjugate No: 1:

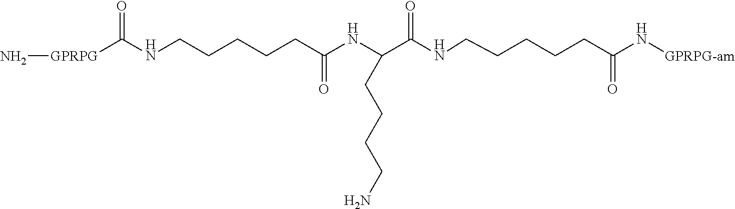

Peptide Conjugate No. 2:

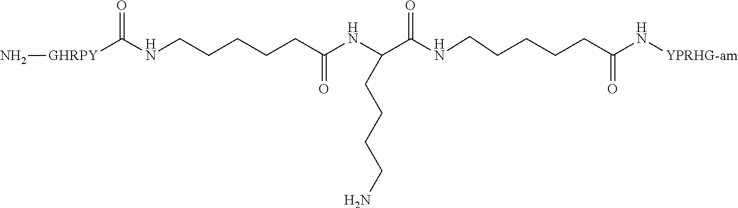

Peptide Dendrimer No. 3:

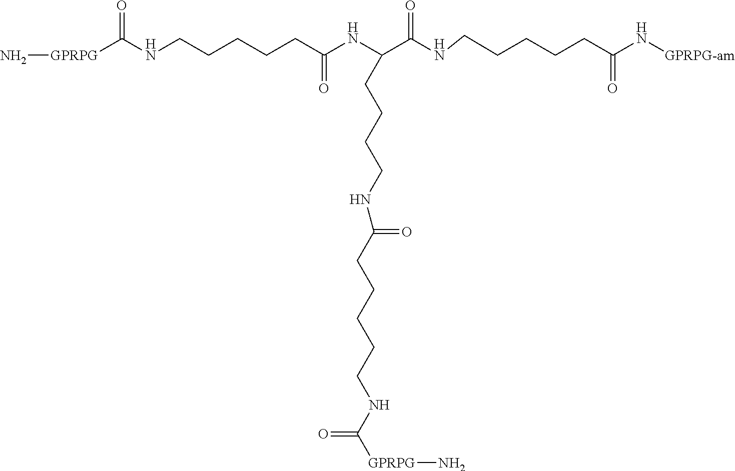

Peptide Dendrimer No. 4:

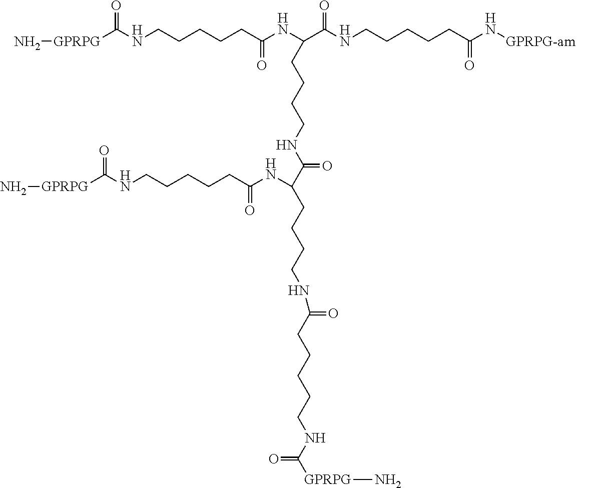

Peptide Dendrirner No. 5:

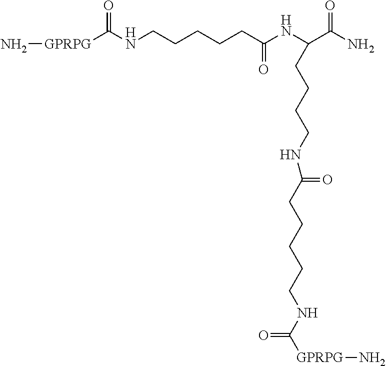

Peptide Dendrimer No. 8:

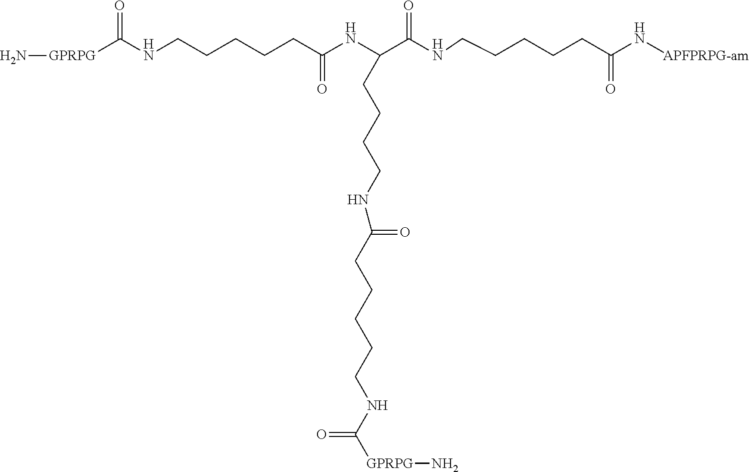

Peptide Dendrirner No. 9:

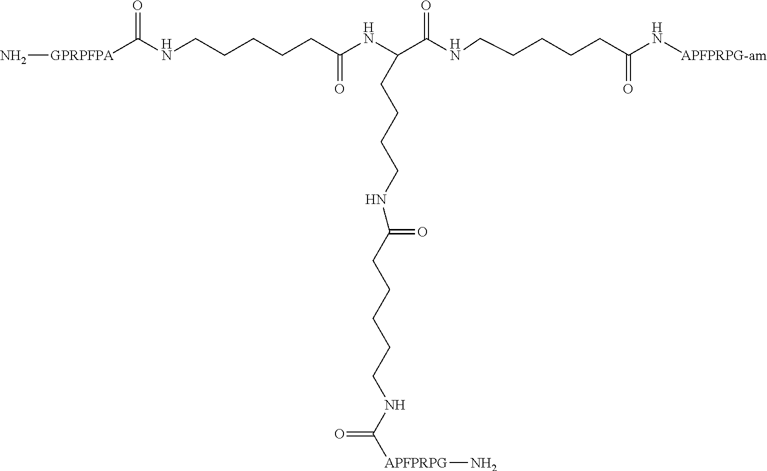

Peptide Dendrimer No. 10:

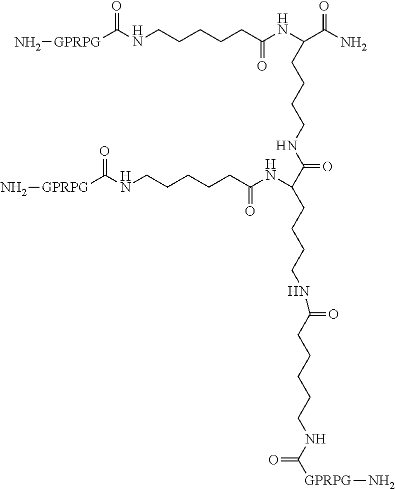

Peptide Dendrimer No. 11

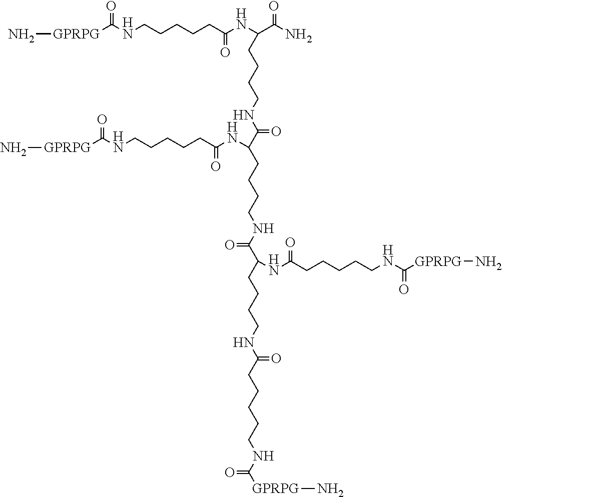

Peptide Dendrimer No. 12:

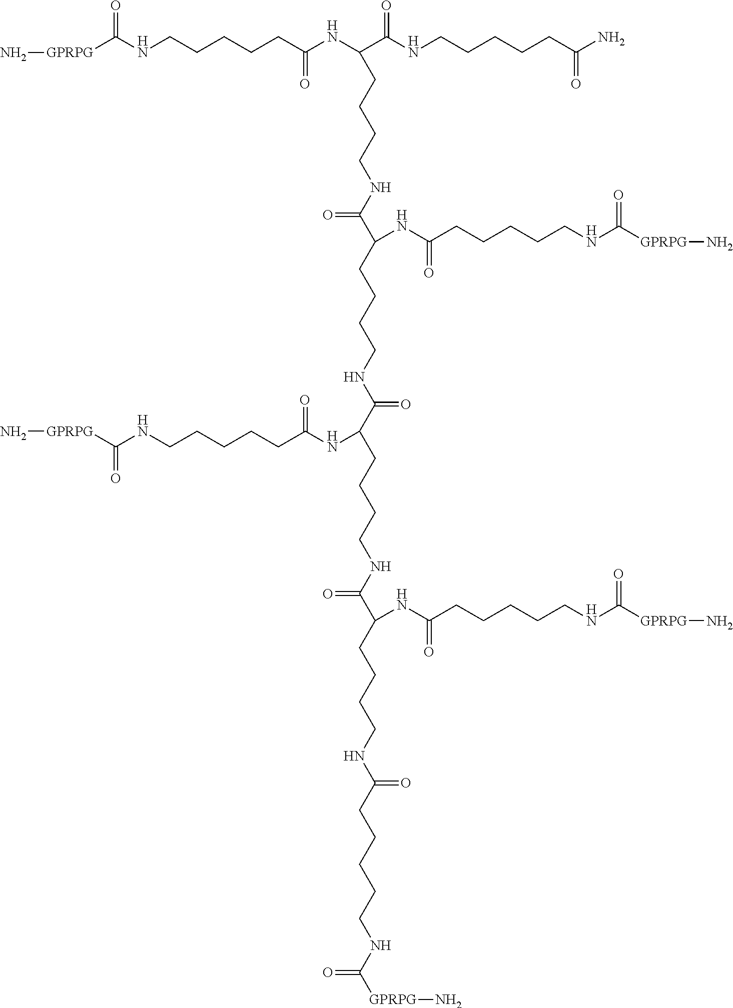

Peptide Dendrimer No. 13:

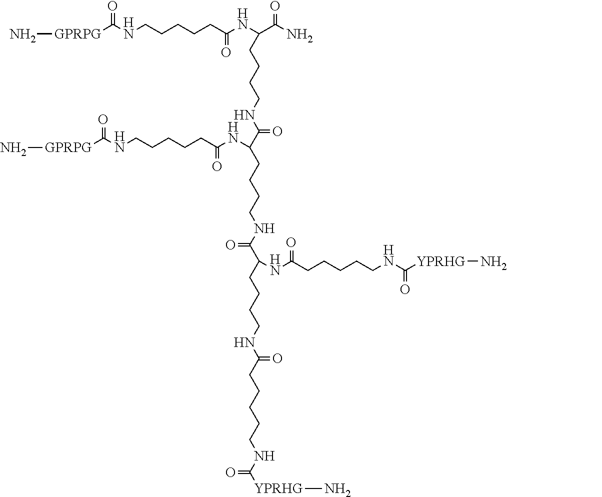

### EXAMPLE 12

### Co-Polymerisation of a Peptide Dendrimer With Fibrinogen

Dendrimer No. 12 comprises a branched core with five consecutive lysine residues. The lysine residues are covalently linked through a side chain of an adjacent lysine residue.

The ability of Peptide Dendrimer No. 12 to polymerise fibrinogen was assessed. 30 μl of dendrimer in solution, at concentration ranging from 0.005-2 mg/ml, was added to 100 μl purified human fibrinogen at 3 mg/ml (the level of fibrinogen found in the blood). Polymerisation of fibrinogen was analysed using a Sigma Amelung KC4 Delta coagulation analyser. FIG. 14 shows a plot of the polymerisation (clotting) times (in se [symbol] increasing concentration of dendrimer.

The results show that the dendrimer was able to copolymerise with fibrinogen almost instantaneously, even at very low concentrations of dendrimer. The increase in clotting time with dendrimer concentrations above 0.5 mg/ml is thought to be explained by an excess of fibrinogen-binding peptides compared to the number of free binding pockets in fibrinogen. At higher concentrations, the fibrinogen-binding peptides of the dendrimer may saturate the fibrinogen binding pockets, resulting in a significant number of excess dendrimer molecules that are not able to copolymerise with fibrinogen.

### EXAMPLE 7

### Effect of Varying the Number of fibrinogen-Binding Peptides Per Dendrimer on the Speed of Copolymerisation With Fibrinogen

This example investigates the effect of varying the number of fibrinogen-binding peptides per peptide dendrimer on the speed of copolymerisation with fibrinogen.

The ability of Peptide Dendrimer Nos. 4, 5, 10, 11, and 12 to copolymerise with fibrinogen was assessed using the same method described in Example 11. The concentration of each dendrimer was varied from 0.005-0.5 mg/ml. FIG. 15 shows a plot of the clotting times (in seconds) with increasing concentration of each different dendrimer.

The results show that dendrimer No. 5 (with only two fibrinogen-binding peptides/dendrimer) was not able to copolymerise with fibrinogen. As the number of fibrinogen-binding peptides was increased from three to five, at concentrations of dendrimer from˜0.125 to˜0.275 mg/ml, the speed of copolymerisation increased. At concentrations below˜0.125 mg/ml dendrimer, dendrimer No. 10 (with three fibrinogen-binding peptides/dendrimer) produced faster clotting times than dendrimer no. 4 (with four fibrinogen-binding peptides/dendrimer). In the range˜0.02-0.5 mg/ml, dendrimer no. 12 (with five fibrinogen-binding peptides/dendrimer) produced almost instantaneous clotting. In the range˜0.05-0.3 mg/ml, dendrimer no. 11 (with four fibrinogen-binding peptides/dendrimer) also produced almost instantaneous clotting.

It is concluded that the speed at which fibrinogen is polymerised by a dendrimer of the invention generally increases as the number of fibrinogen-binding peptides per dendrimer is increased.

### EXAMPLE 13

### Effect of Fibrinogen-Binding Peptide Orientation, and of Different Fibrinogen-Binding Peptide Sequences on Speed of Copolymerisation With Fibrinogen

To assess whether the orientation of a fibrinogen-binding peptide could affe [symbol] a peptide dendrimer to copolymerise with fibrinogen, peptide dendrimers comprising three fibrinogen-binding peptides attached to a single tri-functional amino acid residue (lysine) were synthesised (referred to as ‘three-branch’ dendrimers), but with one of the fibrinogen-binding peptides orientated with its amino-terminal end attached to the branched core, and amidated at its carboxy-terminal end. The ability of peptide dendrimers comprising different fibrinogen-binding peptide sequences to copolymerise with fibrinogen was also tested.

The fibrinogen-binding peptides of Peptide Dendrimer Nos. 3 and 10 are each of sequence GPRPG (SEQ ID NO: 18). Each fibrinogen-binding peptide of Peptide Dendrimer No. 10 is orientated with its carboxy-terminal end attached to the branched core. One of the fibrinogen-binding peptides of Peptide Dendrimer No. 3 is orientated with its amino-terminal end attached to the branched core. The carboxy-terminal end of that peptide comprises an amide group.

Two of the fibrinogen-binding peptides of Peptide Dendrimer No. 8 are of sequence GPRPG (SEQ ID NO: 18), and the third fibrinogen-binding peptide is of sequence APFPRPG (SEQ ID NO: 2) orientated with its amino-terminal end attached to the branched core. The carboxy-terminal end of that peptide comprises an amide group.

Two of the fibrinogen-binding peptides of Peptide Dendrimer No. 9 are of sequence GPRPFPA (SEQ ID NO: 7), and the third fibrinogen-binding peptide is of sequence APFPRPG (SEQ ID NO: 12) orientated with its amino-terminal end attached to the branched core. The carboxy-terminal end of that peptide comprises an amide group.

The sequence GPRPG (SEQ ID NO: 15) binds to hole ‘a’ and hole ‘b’ of fibrinogen, but with some preference for hole ‘a’. The sequence GPRPFPA (SEQ ID NO: 7) binds with high preference for hole ‘a’ in fibrinogen. The sequence Pro-Phe-Pro stabilizes the backbone of the peptide chain and enhances the affinity of the knob-hole interaction (Stabenfeld et al., BLOOD, 2010, 116: 1352-1359).

The ability of the dendrimers to copolymerise with fibrinogen was assessed using the same method described in Example 11, for a concentration of each dendrimer ranging from 0.005-0.5mg/ml. FIG. 16 shows a plot of the clotting times (in seconds) obtained with increasing concentration of each different dendrimer.

The results show that changing the orientation of one of the fibrinogen-binding peptides of a three-branch dendrimer, so that the peptide is orientated with its amino-terminal end attached to the branched core (i.e. Dendrimer No. 3), reduced the ability of the dendrimer to copolymerise with fibrinogen (compare the dotting time of Dendrimer No. [symbol] Dendrimer No. 10). However, at higher fibrinogen concentrations, Dendrimer No. 3 was able to copolymerise with fibrinogen (data not shown).

A three-branch dendrimer with a fibrinogen-binding peptide of different sequence orientated with its amino-terminal end attached to the branched core was able to copolymerise with fibrinogen (see the results for Dendrimer No. 8).

A three-branch dendrimer in which two of the fibrinogen-binding peptides comprise sequence that binds preferentially to hole ‘b’ in fibrinogen (sequence GPRPFPA (SEQ ID NO: 7)), with these peptides orientated with their carboxy-terminal end attached to the branched core, and the other peptide comprising the reverse sequence (i.e. sequence APFPRPG (SEQ ID NO: 2)) orientated with its amino-terminal end attached to the branched core (Dendrimer No. 9) was also very active in copolymerising with fibrinogen.

EXAMPLE 14

### Ability of Peptide Dendrimers With Different Fibrinogen-Binding Peptide Sequences to Copolymerise With Fibrinogen

The GPRPG (SEQ ID NO: 18) and GPRPFPA (SEQ ID NO: 8) motifs primarily bind to the ‘a’ hole on fibrinogen. This example describes an assessment of the ability of a chimeric peptide dendrimer (i.e. a peptide dendrimer with different fibrinogen-binding peptide sequences attached to the same branched core) to copolymerise with fibrinogen.

Peptide dendrimer No. 13 is a chimeric four-branch peptide dendrimer comprising two fibrinogen-binding peptides with sequence GPRPG-(SEQ ID NO: 18) (which has a binding preference for the ‘a’ hole), and two fibrinogen-binding peptides with sequence GHRPY-(SEQ ID NO: 15) (which binds preferentially to the ‘b’ hole). Non-chimeric peptide dendrimers Nos. 11 and 12 are four- and five-arm peptide dendrimers, respectively. Each fibrinogen-binding peptide of these dendrimers has the sequence GPRPG-(SEQ ID NO: 18). Each fibrinogen-binding peptide of Dendrimers Nos. 11, 12, and 13 is attached at its carboxy-terminal end to the branched core.

The ability of the dendrimers to copolymerise with fibrinogen was assessed using the same method described in Example 11, for a concentration of each dendrimer ranging from 0.005-0.5 mg/ml. FIG. 17 shows a plot of the clotting times (in seconds) obtained with increasing concentration of each different dendrimer.

The results show that the clotting speed using the chimeric dendrimer was slower than the non-chimeric dendrimers at concentrations below 0.3 mg/ml. However, FIG. 18 shows a photograph of the hydrogels obtained using the different dendrimers, The g [symbol] with the number of the peptide dendrimer used (11, 12, and 13), and “P” labels a hydrogel formed using a product in which several fibrinogen-binding peptides are attached to soluble human serum albumin. The hydrogel formed by the chimeric dendrimer was more dense and contained less fluid compared to the hydrogels formed using dendrimers Nos. 11 and 12 (at 3 mg/ml fibrinogen, or at higher concentrations of fibrinogen). Thus, although the clotting time was slower using the chimeric dendrimer, the hydrogel formed using this dendrimer was more dense.

### EXAMPLE 15

### Ability of Mixtures of Peptide Dendrimers and Peptide Conjugates to Copolymerise with Fibrinogen

Fibrinogen-binding peptide of sequence GPRP-(SEQ ID NO: 5) binds strongly and preferentially to the ‘a’ hole of fibrinogen (Laudano et al., 1978 PNAS 7S). Peptide Conjugate No. 1 comprises two fibrinogen-binding peptides with this sequence, each attached to a lysine residue. The first peptide is attached its carboxy-terminal end by a linker to the lysine residue, and the second peptide is attached at its amino-terminal end by a linker to the lysine residue. The carboxy-terminal end of the second peptide comprises an amide group.

Fibrinogen-binding peptide of sequence GHRPY-(SEQ ID NO: 16) binds strongly and preferentially to the ‘b’ hole of fibrinogen (Doolittle and Pandi, Biochemistry 2006, 46, 2657-2667). Peptide Conjuoate No. 2 comprises a first fibrinogen-binding peptide with this sequence, attached at its carboxy-terminal end by a linker to a lysine residue. A second fibrinogen-binding peptide, which has the reverse sequence (YPRHG (SEQ ID NO: 19)), is attached at its amino terminal end by a linker to the lysine residue. The carboxy-terminal end of the second peptide comprises an amide group.

The linker allows the peptides to extend away from each other.

Peptide Conjugate No. 1 or 2 (2 mg/ml) was mixed with Peptide Dendrimer No. 3 or 4, and fibrinogen, and the ability of the mixtures to copolymerise with fibrinogen was assessed using the same method described in Example 11, for a concentration of each dendrimer ranging from 0.025-0.5 mg/ml. FIG. 15 shows a plot of the clotting times (in seconds) obtained with increasing concentration of each different dendrimer.

The results show that, surprisingly, only mixtures containing Peptide Conjugate No. 2 (i.e. with the B-knob peptides) and the dendrimer peptides were synergistic and increased activity, whereas mixtures containing the Peptide Conjugate No. 1 (the A-kn [symbol] were not active when added to either Peptide Conjugate No. 2 or the peptide dendrimers.

### EXAMPLE 16

### Ability of Peptide Dendrimers to Polymerise Fibrinogen in Human Plasma

The ability of several different peptide dendrimers (Nos. 4, 5, 8, 9, 10, 11, 12, 13) to polymerise fibrinogen in human plasma was tested.

30 μL of each dendrimer (at a concentration of 0.25 mg/ml) was added to 100 μL human plasma at 37° C., and polymerisation of fibrinogen was determined using a Sigma Amelung KC4 Delta coagulation analyzer.

The clotting times for each dendrimer are shown in FIG. 20, and show that peptide dendrimers Nos. 10, 11, 4, 12 and 13 were able to polymerise fibrinogen in human plasma, with dendrimer No. 12 being particularly effective (with a clotting time of less than one second). However, peptide dendrimers Nos. 5, 8, and 9 were not able to polymerise fibrinogen in human plasma.

### EXAMPLE 12

### Effect of Sterilisation on Ready-to-Use Peptide Dendrimer Formulations

This example describes the effect of Gamma irradiation on the haemostatic activity of peptide dendrimers formulated as a ready-to-use paste with hydrated gelatin.

2 ml of solution of Peptide Dendrimer No. 12 or 13 was mixed with SURGIFLO Haemostatic Matrix (a hydrated flowable gelatin matrix) to form a paste of each peptide. Each paste was sterilised by irradiation with ⁶⁰Co gamma rays at a dose of 30 kGy, and then stored at room temperature. Samples of the sterilised pastes were used for testing after storage for two and four weeks.

After storage, peptide dendrimers were extracted from each paste using 10 mM HEPES buffer. 30 μL of each extract (with a peptide concentration of about 0.25 mg/ml) was added to 100 μL of human fibrinogen at 3 mg/ml, and the ability of each dendrimer to polymerise fibrinogen (the ‘clotting’ activity) at 37° C. was determined using a Sigma Amelung KC4 Delta coagulation analyser. The polymerisation activity of non-irradiated control samples was also determined. The results are summarized in the Table below.

| | Clotting activity (seconds) | | |
|---|---|---|---|
| Peptide | | Storage for 2 | Storage for 4 |
| dendrimer | Non-irradiated | weeks post | weeks post |
| no. | control | irradiation | irradiation |
| 12 | 1 | 1 | 1 |
| 13 | 4.3 | 9.4 | 10 |

The results show that peptide dendrimers of the invention, formulated as a ready-to-use paste with hydrated gelatin, retain ability to polymerise fibrinogen after sterilization by irradiation.

1. A haemostatic material comprising an oxidised cellulose substrate covalently immobilised to a plurality of fibrinogen-binding peptides.

2. The material according to claim 1, in which each peptide is immobilised to the substrate via a carbonyl group of the substrate.

3. The material according to claim 1, in which each peptide is immobilised to the substrate via a spacer.

4. The material according to claim 3, wherein the spacer is covalently linked to the peptide via an amide bond.

5. The material according to claim 1, wherein the spacer is covalently linked to the substrate via an amide bond.

6. The material according to claim 3, in which the spacer comprises a peptide spacer.

7. The material according to claim 3, in which the spacer comprises a non-peptide spacer.

8. The material according to claim 7, wherein the non-peptide spacer comprises a straight chain, preferably wherein the non-peptide spacer comprises the group —(CH₂)ₐ—, wherein a is 1-20, preferably 1-15, 1-10, 1-5, or 2-4.

9. The material according to any preceding claim 1, in which each peptide is immobilised to the substrate via the C-terminus of the peptide.

10. The material according to claim 1, comprising the following structure: [symbol]
—CO—NH— [symbol] —NH—CO— [symbol] —NH₂
where [symbol] =fibrogen-binding peptide.

11. The material according to claim 1, in which each peptide is immobilised to the substrate via the N-terminus of the peptide.

12. The material according to claim 1, comprising the following structure: [symbol]
—CO—NH— [symbol] —CO—NH— [symbol] —COOH
where [symbol] =fibrinogen-binding peptide.

13. The material according to claim 1, in which each fibrinogen-binding peptide comprises the sequence Gly-(Pro/His)-Arg-Xaa (SEQ ID NO: 1) where Xaa is any amino acid and Pro/His means that either proline or histidine is present at that position.

14. The material according to claim 1, in which each fibrinogen-binding peptide is 4-60 residues in length.

15. The material according to claim 1, in which the haemostatic material is in the form of a wound dressing.

16. A method of making a haemostatic material comprising covalently immobilising a plurality of fibrinogen binding peptides to an oxidised cellulose substrate.

17. The method according to claim 16, comprising:
providing a plurality of moieties, each moiety comprising a fibrinogen-binding peptide and a first reactive group in the form of a carboxyl-reactive group;
providing an oxidised cellulose substrate comprising a plurality of second reactive groups in the form of carboxyl groups; and
reacting the first reactive groups with the second reactive groups to covalently immobilise each peptide to the substrate.

18. The method according to claim 17, in which the first reactive group is an amino group.

19. The method according to claim 17, in which each moiety comprises a non-peptide portion which provides the first reactive group.

20. The method according to claim 19, in which the non-peptide portion of each moiety is covalently linked to the α-carbonyl group via the C-terminus of the peptide.

21. The method according to claim 19, in which the non-peptide portion is covalently linked to the peptide via an amide bond.

22. The method according to claim 19, in which the non-peptide portion of each moiety comprises a straight chain group of formula —(CH₂)ₐ—, wherein a is 1-20, preferably 1-15, 1-10, 1-5, or 2-4.

23. The method according to claim 19, in which each moiety comprises the following structure:
H₂N—(CH₂)ₐ—NH—CO— [symbol] —NH₂
where a=1-20, preferably 1-15, 1-10, 1-5, or 2-4; and where [symbol] =fibrinogen-binding peptide.

24. The method according to claim 17, in which each moiety is protected by one or more protecting groups, such that only the first reactive group is capable of reacting with the second reactive group.

25. The method according to claim 16, in which the substrate has been modified by reacting carboxyl groups on the substrate with modifying groups to form spacers on the substrate, in which the second reactive groups are positioned at the end of the spacers.

26. The method according to claim 16, comprising modifying the substrate by reacting carboxyl groups on the substrate with modifying groups to form spacers on the substrate, in which the second reactive groups are positioned at the end of the spacers.

27. The method according to claim 25, in which each modifying group has a first reactive group which is a carboxyl-reactive group, preferably an amino group, and a second reactive group which is a carboxyl group, and the first reactive group is capable of reacting with carboxyl groups on the substrate to form an amide bond.

28. The method according to claim 27, in which the modifying group comprises a peptide.

29. The method according to claim 16, comprising:
providing a plurality of moieties, wherein each moiety comprises a fibrinogen-binding peptide and a first reactive group;
providing a modified substrate comprising a plurality of second reactive groups, in which the second reactive groups are formed by modifying carboxyl groups of the oxidised cellulose substrate; and
reacting the first reactive groups with the second reactive groups.

30. The method according to claim 29, in which the first reactive group is a carboxyl group, preferably the carboxyl group at the C-terminal end of the peptide.

31. The method according to claim 29, in which the second reactive group is a carboxyl-reactive group, preferably an amino group.

32. The method according to claim 29, in which the carboxyl groups on the substrate have been modified by reacting the carboxyl groups with modifying groups, preferably to form spacers on the substrate, in which the second reactive groups are positioned at the end of the spacers.

33. The method according to claim 29, comprising modifying the carboxyl groups on the substrate by reacting the carboxyl groups with modifying groups, preferably forming spacers on the substrate, in which the second reactive groups are positioned at the end of the spacers.

34. The method according to claim 32 in which each modifying group comprises a first carboxyl reactive group and a second carboxyl reactive group, wherein the first and second carboxyl reactive groups are preferably amino groups.

35. The method according to claim 34, in which each modifying group comprises the following structure:
H₂N—(CH₂)ₐ—NH₂
where a is 1-20, preferably 1-15, 1-10 or 1-6.

36. The method according to claim 29, in which each spacer is covalently linked to the substrate by amide bonds.

37. The method according to claim 29, in which the modified substrate comprises the following structure: [symbol]
—CO—NH— [symbol] —NH₂

38. The method according to claim 16, in which each fibrinogen-binding peptide comprises the sequence Gly-(Pro/His)-Arg-Xaa (SEQ ID NO: 1) where Xaa is any amino acid and Pro/His means that either proline or histidine is present at that position.

39. A method of controlling bleeding comprising administering the haemostatic agent according to claim 1, to a wound.

40. A method of covalently immobilising a peptide to a substrate, comprising:
providing a moiety comprising a peptide and a first reactive group in the form of a carboxyl-reactive group linked via the C-terminus of the peptide;
providing a substrate comprising a second reactive group in the form of a carboxyl group; and
reacting the first reactive group with the second reactive group to covalently immobilise each peptide to the substrate, such that the peptide is covalently immobilised to the substrate via its C-terminus.

41. The method according to claim 40, in which the first reactive group is an amino group.

42. The method according to claim 40, in which each moiety comprises a non-peptide portion which provides the first reactive group.

43. The method according to claim 40, in which the non-peptide portion is covalently linked to the peptide by an amide bond.

44. The method according to claim 40, in which the non-peptide portion of the moiety comprises a straight chain group of formula —(CH₂)ₐ—, wherein a is 1-20, preferably 1-15, 1-10, 1-5, or 2-4.

45. The method according to claim 40, in which the moiety comprises the following structure:
H₂N-(CH₂)ₐ—NH—CO— [symbol] —NH₂
where a=1-20, preferably 1-15, 1-10, 1-5, or 2-4.

46. The method according to claim 40, in which the substrate has been modified by reacting carboxyl groups on the substrate with modifying groups to form spacers on the substrate, in which the second reactive groups are positioned at the end of the spacers.

47. The method according to claim 46, in which each modifying group has a first reactive group which is a carboxyl-reactive group, preferably an amino group, and a second reactive group which is a carboxyl group, and the first reactive group is capable of reacting with carboxyl groups on the substrate to form an amide bond.

48. The method according to claim 47, in which the modifying group comprises a peptide.

49. A method of covalently immobilising a peptide to a substrate, comprising:
providing a moiety comprising a peptide and a first reactive group, in which the first reactive group is the carboxyl group at the C-terminus of the peptide, or in which the first reactive group is linked via the C-terminus of the peptide;
providing a modified substrate comprising a second reactive group formed by modifying a carboxyl group of the substrate; and
reacting the first reactive group with the second reactive group to covalently immobilise the peptide to the substrate, such that the peptide is covalently attached to the substrate via its C-terminus.

50. The method according to any of claim 49, in which the second reactive group is a carboxyl-reactive group, preferably an amino group.

51. The method according to claim 49, in which the carboxyl group on the substrate has been modified by reacting the carboxyl group with a modifying group, preferably to form a spacer on the substrate, in which the second reactive group is positioned at the end of the spacer.

52. The method according to claim 49 in which the modifying group comprises a first carboxyl reactive group and a second carboxyl reactive group, wherein the first and second carboxyl reactive groups are preferably amino groups.

53. The method according to claim 52, in which the modifying group comprises the following structure:
H₂N—(CH₂)ₐ—NH₂
where a is 1-20, preferably 1-15, 1-10 or 1-6.

54. The method according to claim 49, in which the spacer is covalently linked to the substrate by an amide bond.

55. The method according to claim 49, in which the modified substrate comprises the following structure: [symbol]
—CO—NH— [symbol] —NH₂

56. The method according to claim 40, in which the peptide is a fibrinogen-binding peptide.

57. The method according to claim 40, in which the substrate comprises oxidised cellulose.

58. The method according to claim 40, in which the substrate is a wound dressing.

59. The method according to claim 40, in which the moiety is protected by one or more protecting groups, such that only the first reactive group is capable of reacting with the second reactive group.

60. An oxidised cellulose substrate covalently immobilised to peptide, in which the peptide is covalently immobilised via its C-terminus.

61.-62. (canceled)

63. The method according to claim 49, in which the peptide is a fibrinogen-binding peptide.

64. The method according to claim 49, in which the substrate comprises oxidised cellulose.

65. The method according to claim 49, in which the substrate is a wound dressing.

66. The method according of claim 49, in which the moiety is protected by one or more protecting groups, such that only the first reactive group is capable of reacting with the second reactive group.


SEQUENCE LISTING (19 sequences)

SEQ ID NO: 1 - protein, 4 aa, Homo sapiens
  MISC_FEATURE (2)..(2): Pro or His
  MISC_FEATURE (4)..(4): Any amino acid
GXRX

SEQ ID NO: 2 - protein, 7 aa, Homo sapiens
APFPRPG

SEQ ID NO: 3 - protein, 4 aa, Homo sapiens
  MISC_FEATURE (4)..(4): Any amino acid, preferably any amino acid other than Val, preferably Pro, Sar or Leu
GPRX

SEQ ID NO: 4 - protein, 4 aa, Homo sapiens
  MISC_FEATURE (4)..(4): Any amino acid other than Pro
GHRX

SEQ ID NO: 5 - protein, 4 aa, Homo sapiens
GPRP

SEQ ID NO: 6 - protein, 4 aa, Homo sapiens
GPRV

SEQ ID NO: 7 - protein, 7 aa, Homo sapiens
GPRPFPA

SEQ ID NO: 8 - protein, 7 aa, Homo sapiens
GPRVVAA

SEQ ID NO: 9 - protein, 8 aa, Homo sapiens
GPRPVVER

SEQ ID NO: 10 - protein, 6 aa, Homo sapiens
GPRPAA

SEQ ID NO: 11 - protein, 7 aa, Homo sapiens
GPRPPEC

SEQ ID NO: 12 - protein, 7 aa, Homo sapiens
GPRPPER

SEQ ID NO: 13 - protein, 6 aa, Homo sapiens
GPSPAA

SEQ ID NO: 14 - protein, 4 aa, Homo sapiens
GHRP

SEQ ID NO: 15 - protein, 5 aa, Homo sapiens
GHRPY

SEQ ID NO: 16 - protein, 5 aa, Homo sapiens
GHRPL

SEQ ID NO: 17 - protein, 5 aa, Homo sapiens
  MISC_FEATURE (5)..(5): carbox-termonal end comprises an amide group
GHRPY

SEQ ID NO: 18 - protein, 5 aa, Homo sapiens
GPRPG

SEQ ID NO: 19 - protein, 5 aa, Homo sapiens
YPRHG